(19) United States
(12) Patent Application Publication
(10) Pub. No.: US 2011/0250134 A1
(43) Pub. Date: Oct. 13, 2011

(54) SUSTAINED RELEASE NITRIC OXIDE FROM LONG LIVED CIRCULATING NANOPARTICLES

(76) Inventors: Pedro Cabrales, San Diego, CA (US); Adam Friedman, Manhattan, NY (US); Joel M. Friedman, South Orange, NJ (US)

(21) Appl. No.: 13/086,346

(22) Filed: Apr. 13, 2011

Related U.S. Application Data
(60) Provisional application No. 61/323,832, filed on Apr. 13, 2010.

(51) Int. Cl. A61K 51/06; A61K 38/48; A61K 38/49; A61K 39/395; A61K 38/14; A61K 9/14; A61P 9/00; A61P 9/10; A61P 7/02; A61P 25/00; A61P 25/18; A61P 35/00; A61P 29/00; A61P 23/02; A61P 31/00; A61P 37/06; A61K 31/722; B82Y 5/00

(52) U.S. Cl. 424/1.73; 514/55; 424/94.3; 424/178.1; 514/20.9; 424/400; 977/773; 977/795

(57) ABSTRACT

The invention provides methods for delivering a NO molecule or precursor thereof to a vascular compartment of a subject comprising: administering to the subject, a NO particle formulation, via an intravascular, intraperitoneal, or intramuscular administration, in an amount sufficient to induce vascular smooth muscle relaxation thereby delivering the NO molecule or precursor thereof to the vascular compartment of the subject, wherein the NO particle formulation comprises NO attached to a nanoparticle or microparticle.

This patent application claims the benefit of the filing dates of U.S. Ser. No. 61/323,832, filed Apr. 13, 2010, the contents of all of which are herein incorporated by reference in their entireties into the present patent application.

Throughout this application various publications are referenced. The disclosures of these publications in their entireties are hereby incorporated by reference into this application in order to more fully describe the state of the art to which this invention pertains.

### BACKGROUND OF THE INVENTION

Nitric oxide (NO) is a lipophilic, diatomic, free radical which is surprisingly stable and soluble in aqueous solutions when compared to other radical species [1]. Endogenous NO is produced enzymatically via L-arginine conversion to NO by three distinct NO synthase (NOS) pathways [2]. With respect to vascular function, shear stress exerted on the luminal (endothelial) surface stimulates vascular endothelial NOS (eNOS), regulating numerous vascular functions, principally smooth muscle tension [3]. Intravascular NO supplementation has many complications as well as is short lived due to the rapid scavenging rate of NO by hemoglobin, within erythrocytes [4]. Currently, the clinical therapeutic potential of NO has only been exploited via inhaled NO gas from pressurized tanks. While this approach is inconvenient and costly [5], inhaled NO gas is still the preferred and only approved NO treatment for acute pulmonary hypertension [6]. Other alternatives for intravascular NO therapy include formulations based on compounds containing either NO or NO precursors in a stable form, which typically lack the capacity for controlled and sustained delivery [7]. Thus, despite the considerable therapeutic potential of NO, systemic deployment of NO to the bedside has proven very difficult.

The largest family of NO donors currently in use is NONOates, also known as diazeniumdiolates. They are complexes of NO with nucleophiles, for which decomposition rate depends on pH, temperature, and the nature of the nucleophile. [9, 10]

The major limitations of infusion of small particles of biologically active materials are to achieve long circulating half-life, and to control the delivery and the size. Biologically active materials administered in such a controlled fashion into tissue or blood are expected to exhibit decreased toxic side effects compared to when the materials are injected in the form of a solution, and may reduce degradation of sensitive compounds in the plasma. A significant obstacle to the use of injectable particles for drug delivery is the rapid clearance of the materials from the blood stream by the macrophages by the reticuloendothelial system.

NO is a neurotransmitter, a macrophage-derived host-defense molecule, an inhibitor of platelet aggregation and endothelium adhesion molecule expression, an antioxidant, cardiac chronotropic, and a potent vasodilator. As vasodilator, NO is synthesized from L-arginine by NO synthase enzymes in endothelial cells, in a process regulated by local condition. When NO diffuses into the smooth muscle cells, it initiates a signaling cascade that leads to vasorelaxation. NO has a short half-life in the circulation, which limits its biological activity. Nitroglycerin is an organic nitrate and clinically approved NO donor. The release of NO from nitroglycerin is short acting and involves specific enzymes and reducing agents. In other NO donors, the NO is bound to a nucleophile, that spontaneous decomposes, liberating NO. The NO release, by nitroglycerin and other NO donors, is not determined by the local conditions, resulting in harmful or ineffective NO levels, as their optimal dose is difficult to determine.

Our discovery involves intravascular exogenous supplementation of NO. Given the critical importance of NO in regulating pathophysiologic states involving the vasculature, controlled and sustained intravascular NO delivery could profoundly impact current treatment of many diseases associated with NO deprivation e.g., cardiovascular disease.

### SUMMARY OF THE INVENTION

The invention provides methods for delivering a NO molecule or precursor thereof to a vascular compartment of a subject. In one embodiment the method comprises administering to the subject, a NO particle formulation, via an intravascular, intraperitoneal, or intramuscular administration, in an amount sufficient to induce relaxation of the vascular compartment thereby delivering the NO molecule or precursor thereof to the vascular compartment of the subject, wherein the NO particle formulation comprises NO attached to a nanoparticle or microparticle. Additionally, novel NO particle formulations are provided.

### BRIEF DESCRIPTION OF THE FIGURES

FIG. 1. NO releasing nanoparticle synthesis and morphology. FIG. 1A. Schematic of NO releasing nanoparticle synthesis. The various components are prepared and combined to form a block gel consisting of a silica hydrogel matrix encompassing the other ingredients. The block gel is dried and the nitrite precursor converted to NO via thermal reduction, resulting in the formation of the nanoparticles. FIG. 1B. TEM of NO releasing nanoparticles. The scale bars represent 100 nm, the bottom one representing the lower left and right panes and the upper one for the upper left image.

FIG. 2. Effects in blood pressure after infusion of NO releasing nanoparticles (NO-np), control nanoparticles (Control-np) and NONOates donors. FIG. 2A Infusion of 10 mg/kg (n=5) NO-np and 20 mg/kg (n=5) NO-np. Maximal decrease pressure was measured 90 min after infusion. MetHb levels 4 h after infusion of NO-np were 9±2% for 10 mg/kg and 14±3% for 20 mg/kg, respectively. FIG. 2B. Infusion of 10 mg/kg (n=5) Control-np and 20 mg/kg (n=5) Control-np. No changes in blood pressure were measure after in fusion of Control-np. FIG. 2C Infusion of 10 mg/kg (n=5) DPTA NONOate and 20 mg/kg (n=5) DPTA NONOate. FIG. 2D Infusion of 10 mg/kg DETA NONOate and 20 mg/kg (n=5) DETA NONOate.

FIG. 3. Exhaled NO after infusion of NO releasing nanoparticles (NO-np) and control nanoparticles (Control-np). FIG. 3A. Exhaled NO after infusion of 10 mg/kg and 20 mg/kg (n=5) of NO-np and Control-np. FIG. 3B. Intravascular fluorescence of NO-np and Control-np.

FIG. 4. Microvascular changes and blood chemistry after infusion of NO releasing nanoparticles (NO-np) and control nanoparticles (Control-np). FIG. 4A. Changes in arteriolar diameter after infusion of 10 (white bars) and 20 (gray bars) mg/kg of NO-np (no pattern) and Control-np (pattern upward diagonal). Diameters (μm, mean±SD) at baseline were 61.7±7.9, N=68. N=number of vessels studied. †, P<0.05 to Baseline; ‡, P<0.05 to Control-np at same concentration. FIG. 4B. Changes in arteriolar blood flow after infusion of NO-np and Control-np. Flow (nl/s, mean±SD) at baseline were 12.6±3.8, N=68. †, P<0.05 to Baseline; ‡, P<0.05 to Control-np at same concentration. FIG. 4C. Changes in functional capillary density (FCD) after infusion of NO-np and Control-np. FCD (perfused capillaries/cm⁻¹, mean±SD) at baseline were 106±6, n=20. †, P<0.05 to Baseline; ‡, P<0.05 to Control-np at same concentration.

FIG. 5. Rolling and immobilized leukocytes after infusion of NO releasing nanoparticles (NO-np) and control nanoparticles (Control-np). FIG. 5A. Immobilized leukocytes after infusion of 10 (white bars) and 20 (gray bars) mg/kg of NO-np (no pattern) and Control-np (pattern upward diagonal). 6 animals were used in each group, 6-8 venules were selected in each animal. †, P<0.05 to Baseline; ‡, P<0.05 to Control-np at same concentration. FIG. 5B. Rolling leukocytes after infusion of NO-np and Control-np. Rolling leukocytes were quantified in the same animals and locations as Immobilized. †, P<0.05 to Baseline; ‡, P<0.05 to Control-np at same concentration. FIG. 5C. Immobilized and rolling leukocytes after infusion of 20 mg/kg of NO-np and Control-np. Red arrows point to immobilized leukocytes on the images, other leukocytes in the images are rolling of flowing.

FIG. 6. Blood pressure vascular tone effects of NO-np and Control-np mediated by NO synthase (NOS) and guanylate cyclase (GCM) metabolism. FIG. 6A. Infusion of 10 mg/kg of NO-np (n=5) and Control-np (n=5) during normal physiological condition. Blood pressure (mmHg, mean±SD) at baseline for each group were NO-np: 112±9 (n=5) and Control-np: 114±8 (n=5). Diameters (μm, mean±SD) at baseline for each group were NO-np: 62±10 (N=24) and Control-np: 64±12 (N=24). FIG. 6B. Infusion of 10 mg/kg of NO-np (n=5) and Control-np (n=5) during NO synthase inhibition with L-NAME. Blood pressure (mmHg, mean±SD) at baseline for each group were NO-np: 111±8 (n=5) and Control-np: 110±7 (n=5). Diameters (μm, mean±SD) at baseline for each group were NO-np: 60±12 (N=22) and Control-np: 65±8 (N=20). FIG. 6C. Infusion of 10 mg/kg of NO-np (n=5) and Control-np (n=5) after ODQ. Blood pressure (mmHg, mean±SD) at baseline for each group were NO-np: 110±6 (n=5) and Control-np: 112±7 (n=5). Diameters (μm, mean±SD) at baseline for each group were NO-np: 58±11 (N=26) and Control-np: 62±10 (N=20). FIG. 6D. Infusion of 10 mg/kg of NO-np (n=5) and Control-np (n=5) during phosphodiesterase inhibitor Zaprinast. Blood pressure (mmHg, mean±SD) at baseline for each group were NO-np: 109±7 (n=5) and Control-np: 112±6 (n=5). Diameters (μm, mean±SD) at baseline for each group were NO-np: 64±12 (N=20) and Control-np: 66±10 (N=20).

FIG. 7. Change in mean arterial pressure and arteriolar diameter after infusion of polymerized bovine hemoglobin (PBH 13 g/dl, 10% of blood volume). NO releasing nanoparticles (NO-np) and control nanoparticles (Control-np) were used to treat hypertension and vasoconstriction 10 min after the infusion of PBH. Intravascular fluorescence of NO-np and Control-np. †, P<0.05 to Baseline, ‡, P<0.05 to Control-np and ¶, P<0.05 between 10 mg/kg and 20 mg/kg of NO-np.

FIG. 8. Nitric oxide (NO) synthase (NOS) inhibition during hemorrhagic shock. FIG. 8A. Shock protocol. Hemorrhagic shock was induced withdrawal of 50% of the estimated blood volume (7% body weight) and NOS inhibition with L-NAME was started 10 min after the end of the hemorrhage. FIG. 8B. NOS inhibition effects on mean arterial pressure (MAP) and heart rate (HR) during the hemorrhagic shock. FIG. 8C. NOS inhibition effects on vascular resistance during the hemorrhagic shock. FIG. 8D. NOS inhibition effects on survivability during the hemorrhagic shock. †, P<0.05 compared to baseline; ‡, P<0.05 compared to Vehicle.

FIG. 9. FIG. 9A. Diagram hemorrhagic shock protocol. Hemorrhagic shock was induced withdrawal of 50% of the estimated blood volume (7% body weight). Treatments were administered 10 min after the end of the hemorrhage. FIG. 9B. and FIG. 9C. nitric oxide (NO) supplementation effects on mean arterial pressure (MAP) and heart rate (FIR) during hemorrhagic shock. †, P<0.05 compared to baseline; ‡, P<0.05 compared to Vehicle; §, P<0.05 compared to Control-nps. Time points: Bl, baseline; H, after hemorrhage, T, after treatment, and 30, 60 and 90 min after treatment. MAP (mmHg, mean±SD) for each animal group were as follows: Baseline: Vehicle, 108±7, n=6; Control-ups, 109±8 n=6; NO-ups, 111±8, n ˜6. n=number of animals. HR (bpm, mean±SD) at baseline for each animal group was as follows: Vehicle, 432±32; Control-ups, 436±43; NO-ups, 444±31.

FIG. 10. Nitric oxide (NO) supplementation effects on changes in arteriolar and venular diameter (FIG. 10A. and FIG. 10B.) and blood flow (FIG. 10C. and FIG. 10D.) during hemorrhagic shock. Broken line represents baseline level. Time points: Bl, baseline; H, after hemorrhage, T, after treatment, and 30, 60 and 90 min after treatment. †, P<0.05 compared to baseline; ‡, P<0.05 compared to Vehicle; §, P<0.05 compared to Control-ups. Diameters (μm, mean±SD) in FIGS. 10A (arteriolar) and 10B (venular) for each animal group were as follows: Baseline: Vehicle (arterioles (A): 62.7±8.2, n=26; venules (V): 64.5±6.8, n ˜24); Control-nps (A: 60.5±6.8, n=24; V: 65.7±8.7, n=27); NO-ups (A: 62.0±7.4, n=25, V: 64.5±8.2, n=26). n=number of vessels studied. RBC velocities (mm/s, mean±SD for each animal group were as follows: Baseline: Vehicle (A: 4.3±1.0, V: 2.3±0.9); Control-ups (A: 4.5±0.8; V: 2.4±1.0); NO-ups (A: 4.3±1.0; V: 2.6±0.7). Calculated flows (nl/s, mean±SD) in FIGS. 10C (arteriolar) and 10D (venular) for each animal group were as follows: Baseline: Vehicle (A: 11.7±3.4; V: 6.9±2.2); Control-ups (A: 12.1±3.2; V: 7.1±2.3); NO-nps (A: 12.0±2.8; V: 6.8±2.3).

FIG. 11. FIG. 11A. Nitric oxide (NO) supplementation effects on changes in functional capillary density (FCD) and FIG. 11B. estimated peripheral vascular resistance (PVR) during hemorrhagic shock. Broken line represents baseline level. Estimation of PVR was made using Hagen-Poiseuille equation, with MAP and microvascular blood flow. Time points: Bl, baseline; H, after hemorrhage, T, after treatment, and 30, 60 and 90 min after treatment. †, P<0.05 compared to baseline; ‡, P<0.05 compared to Vehicle; §, P<0.05 compared to Control-ups, FCD (capillaries per cm, mean±SD) for each animal group were as follows: Baseline: Vehicle, 111±9; Control-ups, 112±11; NO-ups, 109±8.

### DETAILED DESCRIPTION OF THE INVENTION

### Formulations of the Invention

The invention provides novel nitric oxide particle formulations for intravenous, intraperitoneal, and/or intramuscular administration. In one embodiment, the formulation comprises a nitric oxide molecule or precursor thereof attached to a particle, such as a nanoparticle or microparticle. In accordance with the practice of the invention, the nitric oxide molecule or precursor thereof maybe encapsulated in a matrix having a chitosan (or an equivalent thereof), a polyethylene glycol (PEG), and a tetra-methoxy-ortho-silicate (TMOS) molecule. Optionally, the formulation may further comprise a non-reducing sugar or starch.

In accordance with the practice of the invention, the chitosan may be at least 50% deacetylated. In another embodiment, the chitosan is at least 80% deacetylated. In a further embodiment, the chitosan is at least 85% deacetylated. In another embodiment, the concentrations of chitosan in the formulation may be in a range of about 0.05 g to 1 g chitosan per 100 mL of formulation. The chitosan may enhance particle stability and control the release of nitric oxide.

Examples of suitable chitosan equivalents include hydrogels, alginates, carrageenans (e.g., sold by Carrington Laboratories, Inc., Irving, Tex.), and starches (e.g., sold by Hospira, Inc., Lake Forest, Ill., B. Braun Melsungen, Melsungen, Germany, and Teva Parenteral Medicines, Inc., Irvine, Calif.). Merely by way of example, hydrogels include a network of polymer chains that are hydrophilic, sometimes found as a colloidal gel in which water is the dispersion medium (e.g., sold by Carrington Laboratories, Inc., Irving, Tex., Medline Industries, Inc., Mundelein, Ill.). Additionally, alginates are anionic polysaccharides also known as alginic acid (e.g., sold by ConvaTec, Ltd., Deeside, UK, Medline Industries, Inc., Mundelein, Ill.).

In one embodiment, the concentration of tetra-methoxy-ortho-silicate in the composition may be from about 0.5 mL to about 5 mL of TMOS per 24 mL of formulation.

In accordance with the practice of the invention, reducing sugars may be any sugar that has a reactive aldehyde or ketone group. The reducing sugar may be used in a reaction to reduce nitrite to nitric oxide, All simple sugars are reducing sugars. Suitable examples of reducing sugars include, but are not limited to, glucose, tagatose, galactose, ribose, fructose, lactose, arabinose, maltose, and maltotriose. Different isomers may be used. For example, D-glucose and/or L-glucose may be used. The concentration of the reducing sugar in the formulation may be about 20 mg to about 100 mg of reducing sugar per milliliter of formulation.

Additionally, the PEG may be a linear PEG, a branched PEG, or a highly branched PEG. Further, the PEG may have a molecular weight in a range of about 200 to about 60,000 daltons molecular weight. For example, the polyethylene glycol may have a molecular weight range of about 200 to 20,000 Da. In another example, the polyethylene glycol may have a molecular weight range of about 400 to 10,000 Da. PEGs of various molecular weights may be obtained, for example from Nektar Therapeutics, Huntsville, Ala. In accordance with the practice of the invention, the concentration of polyethylene glycol in the formulation may be in the range of about 1 to 5 mL of polyethylene glycol per 24 mL of formulation.

Examples of suitable nitric oxide precursors include, but are not limited to, organic nitrites or organic nitrates. In accordance with the practice of the invention, the nitrite may be a monovalent or divalent cation salt of nitrite, including for example, one or more of sodium nitrite, calcium nitrate, potassium nitrite, and/or magnesium nitrate. Suitable concentrations of nitrites and/or nitrates in the formulation may be from about 20 nmol to about one molar.

In accordance with the practice of the invention, the formulation comprises nanoparticles having a range in size of about less than five to about 1000 nanometers (am) in diameter. For example, the nanoparticles may have a size range of about 100 to 800 nm in diameter. In another example, the nanoparticles may have a size range of between 50 nm to 300 nm in diameter. In yet another example, the nanoparticles may have a size range of between 20 nm and 200 nm in diameter. In a further example, the nanoparticles may have a size range of less than about 0.5 micrometers (μm) in diameter. Additionally, the nanoparticles may have a size range of less than about 1 μm in diameter.

Preferably, the formulation comprises particles, the majority of which (e.g. greater than 50%) fall in the size range of less than about five nm to less than about 100 μm in diameter. Additionally, in another preferred embodiment, the majority of the particles of the formulation fall in the size range of about five nm to less than about 100 μM in diameter. In yet another embodiment, the majority of the particles of the formulation fall in the size range of about five nm to less than about 100 nm in diameter. In a further embodiment, the majority of the particles of the formulation fall in the size range of about less than 100 nm in diameter. In yet a further embodiment, the majority of the particles of the formulation fall in the size range of about less than 100 μm in diameter. In a still further embodiment, the formulation contains particles, at least about 30 percent of which are in the size range of about five nm to about 10 nm and about less than 5 percent of which have a size range of least about 250 nm.

Further, in accordance with the practice of the invention, microparticles may have a diameter of from about 1 nm to about 100 μm. For example, the microparticle may have a diameter of from about 2 μm to about 50 μm.

In accordance with the practice of the invention, the nitric oxide particle formulation may further comprise a detection or therapeutic agent bound to the surface of the nanoparticles or microparticles via the terminal hydroxyl group of the particle. For example, the therapeutic agent may be a cytotoxic agent. Examples of suitable cytotoxic agents include, but are not limited to, ricin, doxorubicin, daunorubicin, taxol, ethidium bromide, mitomycin, etoposide, tenoposide, vincristine, Vinblastine, colchicine, dihydroxy anthracin dione, actinomycin D, diphtheria toxin, pseudomonas exotoxin (PE A), PE40, abrin and glucocorticoid.

In another embodiment, the therapeutic agent includes, but is not limited to, streptokinase, tissue plasminogen activator, plasmin, urokinase, tissue factor protease inhibitor, anti-coagulant protein, metalloproteinase inhibitor, anti-inflammatory agent, immunosuppressive agent, steroids, analgesics, local anesthetics, antibiotic agents, chemotherapeutic agents, anti-proliferative agents, anti-mitotic agents, angiogenic agents, antipsychotic agents, central nervous system (CNS) agents, fibrinolytic agents, growth factors, and antibodies.

In accordance with the practice of the invention, the detection agent may be a fluorescent polypeptide, paramagnetic isotope, heavy metal, or a radioisotope. Examples of suitable radioisotopes include, but are not limited to, ²¹²Bi, ¹³¹I, ¹³³In, ⁹⁰Y and ¹⁸⁶Re.

In one embodiment of the invention, the nitric oxide (NO) releasing nanoparticle (NO-np) formulation (also referred to herein as a NO particle formulation) can be injected into the circulation of a subject so as to result in sustained release of NO within the circulation. In another embodiment, the NO particle formulation is locally injected in a specific location in the subject which releases NO in that locale. When in circulation the NO-np formulation e.g., increases the exhaled NO levels, decreases blood pressure, dilates blood vessels, decreases inflammation, extends the circulation time, increases the formation of nitroso compounds and/or enhances phosphodiesterase inhibitors effects, and exhibits one or more of these effects over several hours. Infusion of the particles may counter-act vasoconstriction due to the presence of vasoconstrictive acellular hemoglobin based blood substitutes within the circulation.

The low level infusion of the NO particle formulations of the invention may minimize or reverse both vasoconstrictive and pro-inflammatory properties of a wide variety of infusible therapeutics and may be added thereto including: blood substitutes, whole blood and nanoparticle based drug delivery or imaging materials. Additionally, formulations of the invention can be used to enhance the efficacy of phosphodiesterase inhibitors and other similar therapeutics and thus reduce the dosing for the drugs. Further, the vasodilatory behavior of the NO releasing nanoparticles can be used to increase the efficacy of volume expanders and other vascular support materials by maintaining adequate functional capillary density-a key determinant of transfusion efficacy.

In one embodiment, the formulation includes a hydrogel/glass nanoparticle capable of sustained release of NO. The formulation is based on silane based hydrogel technology, such as those made from tetramethoxysilane (TMOS) (US 2009/0297634). In additional embodiments, the formulation incorporated glass forming materials such as sugars and polysaccharides (e.g., chitosan) into the sol-gel recipe to “plug” the pores of the hydrogel with a relatively stable (when dry) hydrogen bonded network of glass forming molecules and polyethylene glycol (PEG). These additives result in several desirable properties. Upon drying the mixture spontaneously forms nanoparticles. The inclusion of nitrite into the initial mixture results in nanoparticles that release NO when the dry particles are exposed to moisture. Most significantly, the release profile for the NO is easily tuned through manipulation of the components (e.g. molecular weight (MW) of the added PEG) comprising the particles. Additionally the inclusion of other silanes allows for facile manipulation of the internal environment (polarity, hydrophobicity, charge) as well as a basis for derivatizing the surface of particles after formation (e.g. enclusion of thiols or amines).

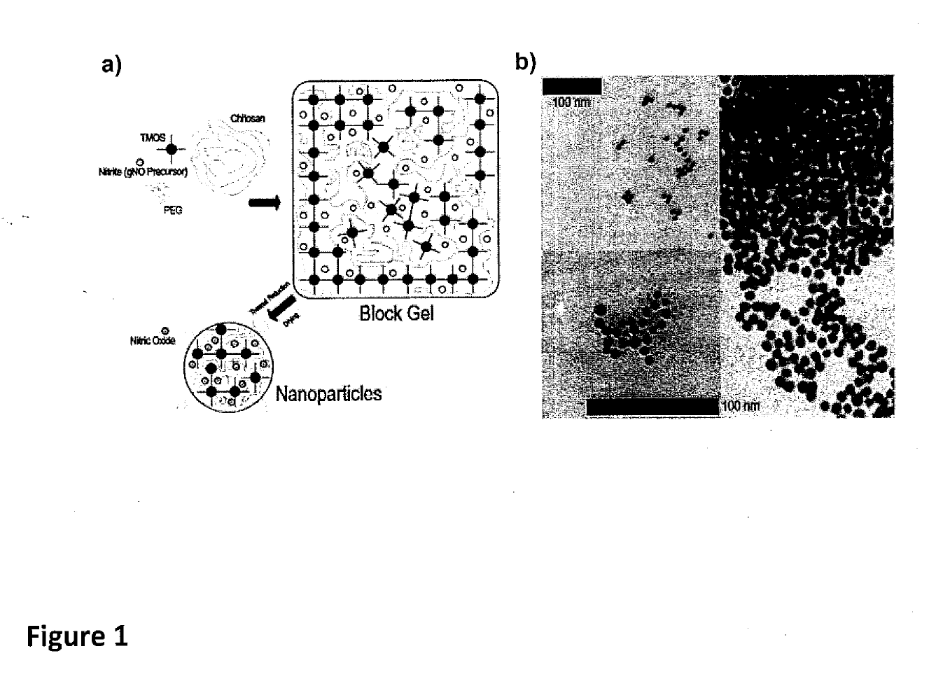

In one embodiment, the formulation utilizes a sol-gel precursor in conjunction with a glass-forming combination of chitosan and polyethylene glycol to form a fine powder comprised of nanoparticles upon drying via lyophilization (FIG. 1A). This hydrogel-glass hybrid material retains NO or an NO precursor in a stable form when dry and releases NO upon exposure to moisture. This release process is controlled by the amount and characteristics of the materials used with drastic differences in NO release kinetics obtainable based on modification of described components [8]. This nanoparticles suffer minimal changes of release properties during long-term dry storage. The nanoparticles, seen on transmission electron microscopy (FIG. 1B), are formed spontaneously and average e.g., 10 nm in diameter. Since a thermally driven redox reaction involving nitrite is used to generate the NO within the particles, the formed NO is retained within the dry particles [8].

In accordance with the practice of the invention, the formulations may be made by the methods as described in Friedman et al., US 2009/0297634.

### Methods of the Invention

The invention provides methods for delivering a nitric oxide molecule or precursor thereof to a vascular compartment. The method comprises administering a nitric oxide particle formulation of the invention, via an intravascular, intraperitoneal, or intro about muscular or intramuscular administration. Preferably, the nitric oxide particle formulation is administered in an amount sufficient to induce vascular smooth muscle relaxation. The nitric oxide formulations of the invention described in the section entitled “Formulations of the Invention” may be used in the methods of the invention.

Additionally, the invention provides methods for enlarging the diameter of blood vessels by delivering a nitric oxide molecule or precursor thereof in the vascular compartment of blood vessels by administering a nitric oxide particle formulation to the subject.

Further, the invention provides methods for increasing profusion and oxygenation of blood vessels by delivering a nitric oxide molecule or precursor thereof in the vascular compartment of blood vessels by administering a nitric oxide particle formulation of the invention to the subject.

Further still, the invention provides methods for decreasing blood pressure in subject by delivering a nitric oxide molecule or precursor thereof to the vascular compartment of blood vessels by administering a nitric oxide particle formulation of the invention to the subject.

Also, the invention provides methods for decreasing macrophage activation in the subject by delivering a nitric oxide molecule or precursor thereof to the vascular compartment of blood vessels by administering in nitric oxide particle formulation of the invention to the subject.

In a further embodiment of the invention, methods for alleviating the symptoms of peripheral vascular disease are provided. The method comprises administering nitric oxide particle formulation of the invention to the subject so as to deliver a nitric oxide molecule or precursor thereof to the vascular compartment of blood vessels.

In accordance with the practice of the invention, the nitric oxide particle formulation may further comprise a detection or therapeutic agent bound to the surface of the nanoparticles or microparticles via the terminal hydroxyl group of the particle. For example, the therapeutic agent may be a cytotoxic agent. Examples of suitable cytotoxic agents include, but are not limited to, ricin, doxorubicin, daunorubicin, taxol, ethidium bromide, mitomycin, etoposide, tenoposide, vincristine, Vinblastine, colchicine, dihydroxy anthracin dione, actinomycin D, diphtheria toxin, pseudomonas exotoxin (PE A), PE40, abrin and glucocorticoid.

In another embodiment, the therapeutic agent includes, but is not limited to, streptokinase, tissue plasminogen activator, plasmin, urokinase, tissue factor protease inhibitor, anti-coagulant protein, metalloproteinase inhibitor, anti-inflammatory agent, immunosuppressive agent, steroids, analgesics, local anesthetics, antibiotic agents, chemotherapeutic agents, anti-proliferative agents, anti-mitotic agents, angiogenic agents, antipsychotic agents, central nervous system (CNS) agents, fibrinolytic agents, growth factors, and antibodies.

In accordance with the practice of the invention, the detection agent may be a fluorescent polypeptide, paramagnetic isotope, heavy metal, or a radioisotope. Examples of suitable radioisotopes include, but are not limited to, ²¹²Bi, ¹³¹I, ¹³¹In, ₉₀Y and ¹⁸⁶Re.

Further, the subject may be any of humans, non-human primates, rodents, guinea pigs, rabbits, sheep, pigs, goats, cows, horses, dogs, cats, birds, and fish.

The invention additionally provides methods for inhibiting, treating, or preventing disease in the subject by delivering nitric oxide in the vascular compartment of blood vessels of the subject by the methods above. Examples of diseases include cardiovascular disease, peripheral vascular disease, erectile dysfunction, and scleroderma. In accordance with the practice of the invention, the cardiovascular disease may cause plaque rupture, plaque erosion, acute coronary syndrome, stroke, transient ischemia, heart attack, angina, unstable angina, thrombosis, myocardial infarction, ischemic heart disease, peripheral artery disease, or transplantation induced sclerosis.

Additionally, the invention provides methods for promoting angiogenesis, wound healing, or vasodilation in the subject comprising ministering to the subject and amount of the nitric oxide particle formulation of the invention effective to promote angiogenesis, wound healing or vasodilation.

For example, the amount of nitric oxide particle formulation so administered may be from about 0.1 to 1000 mg per kilograms of body weight. However, the most effective so mode of administration and dosage regimen for the formulations of the invention depends upon the location, extent, or type of the disease being treated, the severity and course of the medical disorder, the subject's health and response to treatment and the judgment of the treating physician. Accordingly, the dosages of the compositions of the invention should be titrated to the individual subject and/or by the specific medical condition or disease.

The following examples serve to illustrate the present invention. These examples are in no way intended to limit the scope of the invention.

### EXAMPLES

### Example 1

### Materials and Methods

Synthesis of NO-np. The synthesis of NO-np was recently reported.[8] Briefly, a hydrogel/glass composite was synthesized using a mixture of tetramethylorthosilicate, polyethylene glycol, chitosan, glucose, and sodium nitrite in a 0.5 M sodium phosphate buffer (pH 7). The nitrite was reduced to NO within the matrix because of the glass properties of the composite effecting redox reactions initiated with thermally generated electrons from glucose. After redox reaction, the ingredients were combined and dried using a lyophilizer, resulting in a fine powder comprising nanoparticles containing NO. Once exposed to an aqueous environment, the hydrogel properties of the composite allow for an opening of the water channels inside the particles, facilitating the release of the trapped NO over extended time periods.

Transmission electron microscopy. Transmission electron microscopy was used to characterize morphology of the nanoparticles. Samples were mixed with 1% Uranyl Acetate, placed onto a freshly glowed carbon coated grid, and blotted dry. Imaging was performed with a JEOL 100CX II transmission electron microscope (JEOL Ltd., Tokyo, Japan) operating at 80 kV.

Animal Preparation. Investigations were performed in 50-65 g male Golden Syrian Hamsters (Charles River Laboratories, Boston, Mass.) fitted with a dorsal skinfold chamber window. Animal handling and care followed the NIH Guide for the Care and Use of Laboratory Animals. All experimental protocols were approved by the local animal care committee, The hamster chamber window model is widely used for microvascular studies in the unanesthetized state, and the complete surgical technique is described in detail elsewhere. [12]

Inclusion Criteria. Animals were suitable for the experiments if: 1) systemic parameters were within normal range, namely, heart rate (HR)>340 beat/min, mean arterial blood pressure (MAP)>80 mmHg, systemic Hct >45%, and arterial oxygen partial pressure (PaO₂)>50 mmHg; and 2) microscopic examination of the tissue in the chamber observed under 650× magnification did not reveal signs of edema or bleeding. Hamsters are a fossorial species with a lower arterial PO₂ than other rodents due to their adaptation to the subterranean environment.

NO-donors or NO-nanoparticles infusion. Animals were infused with NO releasing agents suspended in deoxygenated saline. Solutions were infused in a volume of 50 μl (equivalent to less than 2% of the animals blood volume) via the jugular vein at a rate of 100 μl/min. The following NO donors (Cayman Chemicals, Ann Arbor, Mich.) were used: DETA-NONOate (Diethylenetriamine NONOate, CAS 146724-94-9) and DPTA-NONOate (Dipropylenetriamine NONOate, CAS 146724-95-0).

Systemic Parameters and Blood Chemistry. MAP and heart rate (HR) were recorded continuously (MP 150, Biopac System; Santa Barbara, Calif.). Hct was measured from centrifuged arterial blood samples taken in heparinized capillary tubes. Hb content was determined spectrophotometrically from a single drop of blood (B-Hemoglobin, Hemocue, Stockholm, Sweden). The methemoglobin (MetHb) level was measured via the cyanomethemoglobin method. Arterial blood was collected in heparinized glass capillaries and immediately analyzed for PaO₂, PaCO₂, base excess (BE) and pH (Blood Chemistry Analyzer 248, Bayer, Norwood, Mass.). The comparatively low PaO₂ and high PaCO₂ of these animals is a consequence of their adaptation to a fossorial environment.

Plasma nitrite/nitrate. Blood samples were collected from carotid artery catheter and centrifuged to separate RBCs and plasma. Plasma proteins were removed by adding equivolume of methanol, and centrifuged at 15000 rpm for 10 min at 4° C. Concentration of nitrite and nitrate in the supernatant were measured with a NOx analyzer (ENO-20; Eicom, Kyoto, Japan). This analyzer combines Griess method and high-performance liquid chromatography.

Exhaled NO. Gaseous NO levels were measured using a chemiluminescent NO analyzer (Sievers NO analyzer, Model 280i, Boulder, Colo.). Pure air at 1 L/min was continuously supplied via a modified sealed tube similar to that used to secure the hamster to the microscope during microvascular studies. 0.2 L/min were introduced into the NO analyzer for one minute every ten minutes, while excess flow was evacuated through an underwater seal (1 cm H₂O). NO calibration gases were introduced at the location where the animals sit during the experiment, to account for reactions between oxygen and NO.

Total NO released (NO agents decomposition). Gaseous NO levels were measured using a chemiluminescent NO analyzer (Sievers NO analyzer, Model 280i, Boulder, Colo.). NO donors or NO-np were added to Di water (5 ml). pH was balanced to 7.4 using Trizma Base and Trizma HCl (Sigma-Aldrich, Saint Louis, Mo.). Solutions were continuously bubbled with pure nitrogen (0.2 L/min). The gas phase was collected into the NO analyzer for a minute every 10 min., and the excess flow was evacuated through an underwater seal (1 cm H₂O).

Microvascular Experimental Setup. The unanesthetized animal was placed in a restraining tube with a longitudinal slit from which the window chamber protruded, then fixed to the microscopic stage of a transillumination intravital microscope (BX51WI, Olympus, New Hyde Park, N.Y.). The animals were given 20 min to adjust to the change in the tube environment before measurements. The tissue image was projected onto a CCD camera (COHU 4815) connected to a video recorder and viewed on a monitor. Measurements were carried out using a 40× (LUMPFL-WIR, numerical aperture 0.8, Olympus) water immersion objective. Microhemodynamic measurements were compared to baseline levels obtained before the experimental procedure. The same vessels and functional capillary fields were followed so that direct comparisons to their baseline levels could be performed allowing for more robust statistics for small sample populations.

Microhemodynamics. Arteriolar and venular blood flow velocities were measured on-line by using the photodiode cross-correlation method (Photo Diode/Velocity Tracker Model 102B, Vista Electronics, San Diego, Calif.). The measured centerline velocity was corrected according to vessel size to obtain the mean RBC velocity (V). A video image-shearing method was used to measure vessel diameter (D). Blood flow (O) was calculated from the measured values as Q=π×V (D/2)². Changes in arteriolar and venular diameter from baseline were used as indicators of a change in vascular tone. This calculation assumes a parabolic velocity profile and has been found to be applicable to tubes of 15-80 μm internal diameters and for Hcts in the range of 6-60%.

Functional Capillary Density (FCD), Functional capillaries, defined as those capillary segments that have RBC transit of at least a single RBC in a 45 s period, in 10 successive microscopic fields were assessed, totaling a region of 0.46 mm². Each field had between two and five capillary segments with RBC flow. FCD (cm⁻¹), i.e., total length of RBC perfused capillaries divided by the area of the microscopic field of view, was evaluated by measuring and adding the length of capillaries that had RBC transit in the field of view. The relative change in FCD from baseline levels after each intervention is indicative of the extent of capillary perfusion.

Leukocyte-endothelium interaction. Intravenous injection of acridine orange (5 mg/kg solution in saline) was used to quantify microvascular leukocytes (adherence and rolling) in post capillary venules (25-50 μm). Low light fluorescent microscopy was used during leukocyte endothelium assessment (ORCA 9247, Hamamatsu, Tokyo, Japan). Briefly, the straight portion of venules was exposed to low intensity epi-illumination for 60 s and digitally video recorded (10 frames/s). Leukocytes were counted during video playback in a 100 μm length segment and categorized according to their flow behavior as “passers,” including “free-flowing” leukocytes, and “rolling” and “immobilized” cells.[13] Measurements of immobilized leukocytes were measured over a period of 120 minutes, as acridine orange toxicity affects leukocyte viability.

Circulating time of NO-np. NO-np were incubated with Rhodamine 6G (20 mg/dl, Sigma-Aldrich. St. Louis) for 30 min, then washed three times and re-suspended in saline, and infused IV as described previously. A photometric assembly in an Olympus BX51WI was used to determine fluorescence of particles in the microvascular window preparation. Briefly, tissue was illuminated (525 nm) using a ALPHA Vivid XF105-2 (Omega Filters, Brattleboro, Vt.) and fluorescence (555 nm) was evaluated by using a photomultiplier (Hamamatsu R928, Tokyo, Japan). Output from the photomultiplier was calibrated in terms of the number of detected photons per unit area of emission.

Drugs NO and Guanylate Cyclase metabolism. NO synthase inhibition was induced by continuous infusion of L-NAME (N-nitro-L-arginine methyl ester, 30 mg/kg, 20 μl/min; Sigma-Aldrich. St. Louis, Mo.). Irreversible heme-site inhibitor of soluble guanylyl cyclase was induced with ODQ (1H-[1,2,4] Oxadiazolo[4,3-a]quinoxalin-1-one; Cayman Chemicals, Aim Arbor, Mich.) by IV infusion of 2 mg/kg 20 min before infusion of NO-np. Irreversible heme-site inhibitor of soluble guanylyl cyclase was induced with ODQ (1H-[1,2,4]Oxadiazolo[4,3-a]quinoxalin-1-one; (Cayman Chemicals, Ann Arbor, Mich.) by IV infusion of 2 mg/kg 20 min before infusion of NO-np.[14] Selective inhibition of cGMP-specific phosphodiesterase was produced by continuous infusion of Zaprinast (2-(o-Propoxyphenyl)-8-azapurin-6-one; CAS 37762-06-4; 0.1 mg/ml/hr). Zaprinast enhances the vasodilatory effects of NO in a range of vascular tissues by prolonging the cGMP-mediated activation of cGMP-dependent protein kinase.[15]

Data analysis. Results are presented as mean standard deviation. Data within each group were analyzed using analysis of variance for repeated measurements (ANOVA, Kruskal-Wallis test). When appropriate, post hoc analyses were performed with the Dunns multiple comparison test. All statistics were calculated using GraphPad Prism 4.01 (GraphPad Software, Inc., San Diego, Calif.). Changes were considered statistically significant if P<0.05.

### Results

Total NO release from NONOate and NO-np. A NONOate theoretically releases 2 moles of NO per mole of parent compound, although experimentally, DETA NONOate liberates 10.7 μmols of NO/mg and DPTA NONOate liberates 9.4 mmols of NO/mg. In contrast to the NONOates, the NO releasing nanoparticle (NO-np) utilizes a novel redox chemistry to generate and capture electrons, reducing nitrite to NO and retaining it within the particles. NO-np half-life in the circulation was found to be approximately 4 hours 37° C., pH 7.4, and one mg of powder is capable of releasing 0.32 mmols of NO.

A total of 110 animals were entered into the study; all animals tolerated all of the protocols without visible signs of discomfort. Four sets of experiments were performed: i) changes in blood gas parameters, systemic hemodynamics (blood pressure and heart rate) and microhemodynamics (microvessel diameter and blood flow); ii) microvascular inflammatory parameters (Leukocyte activation: immobilized and rolling); iii) exhaled NO and circulating time; and, iv) NOS and cGMP metabolism. Five animals were assigned to each experimental group in each set of experiment. All groups were statistically similar (P>0.40) in systemic and microcirculation parameters at baseline.

Blood gas parameters. Table 1 presents summary of blood gas parameters at baseline and after infusion of NO-np and Control-np. No changes in Hct and Hb were measured after infusion of the NO-np or Control-np. Infusion of NO-np increased MetHb levels from baseline after 2 h. MetHb levels 4 h after infusion of NO-np were 9±2% for 10 mg/kg and 14±3% for 20 mg/kg, respectively. Plasma nitrite and nitrate increased from baseline after infusion of NO-np proportional to the dose of NO-np infused at 1 h and 2 h after infusion. Arterial blood pO₂ increased from baseline after infusion of NO-rip.

Arterial blood pH decreased from baseline compared after infusion of NO-np. Consequentially, blood acid-base balance decreased from baseline after infusion of NO-np. No significant change in arterial blood gas parameter was measured after infusion of Control-np.

TABLE 1
Blood chemistry

| | | NO-np | | | | Control-np | | | |
|---|---|---|---|---|---|---|---|---|---|
| | | 10 mg/kg_{BW} | | 20 mg/kg_{BW} | | 10 mg/kg_{BW} | | 20 mg/kg_{BW} | |
| | | Time after infusion, min | | | | | | | |
| | Baseline | 60 | 120 | 60 | 120 | 60 | 120 | 60 | 120 |
| MetHb, % | -o- | | 5 ± 2^{†‡} | | 12 ± 3^{†‡} | | -o- | | -o- |
| NO₂⁻, ηM | 459 ± 48 | 1042 ± 209^{†‡} | 1428 ± 248^{†‡} | 1422 ± 321^{†‡} | 2022 ± 331^{†‡} | 494 ± 46 | 539 ± 62 | 534 ± 54 | 572 ± 71 |
| NO₃⁻, μM | 1.4 ± 0.4 | 2.8 ± 0.9^{†‡} | 3.2 ± 0.8^{†‡} | 3.9 ± 1.0^{†‡} | 4.2 ± 1.1^{†‡} | 1.7 ± 0.4 | 1.8 ± 0.4 | 1.8 ± 0.5 | 1.9 ± 0.6 |
| pₐO₂, mmHg | 57.2 ± 6.6 | 76.2 ± 7.2^{†‡} | 73.1 ± 6.7^{†‡} | 81.4 ± 9.3^{†‡} | 82.5 ± 7.2^{†‡} | 58.4 ± 7.3 | 56.2 ± 7.2 | 59.1 ± 8.1 | 59.8 ± 6.9 |
| pₐCO₂, mmHg | 53.7 ± 5.8 | 47.8 ± 6.8 | 47.7 ± 5.8 | 44.8 ± 7.8 | 43.8 ± 6.9 | 52.3 ± 6.5 | 53.4 ± 6.2 | 51.2 ± 6.0 | 48.9 ± 5.8 |

Values are means ± SD. Baseline included all the animals. MetHb, fraction of Total Hb converted to Methemoglobin; NO₂⁻, Nitrite; NO₃⁻, Nitrate; PaO₂, arterial partial O₂ pressure; PaCO₂, arterial partial pressure of CO₂.
^{†}P < 0.05 compared to Baseline;
^{‡}P < 0.05 compared to control-np at same concentration.

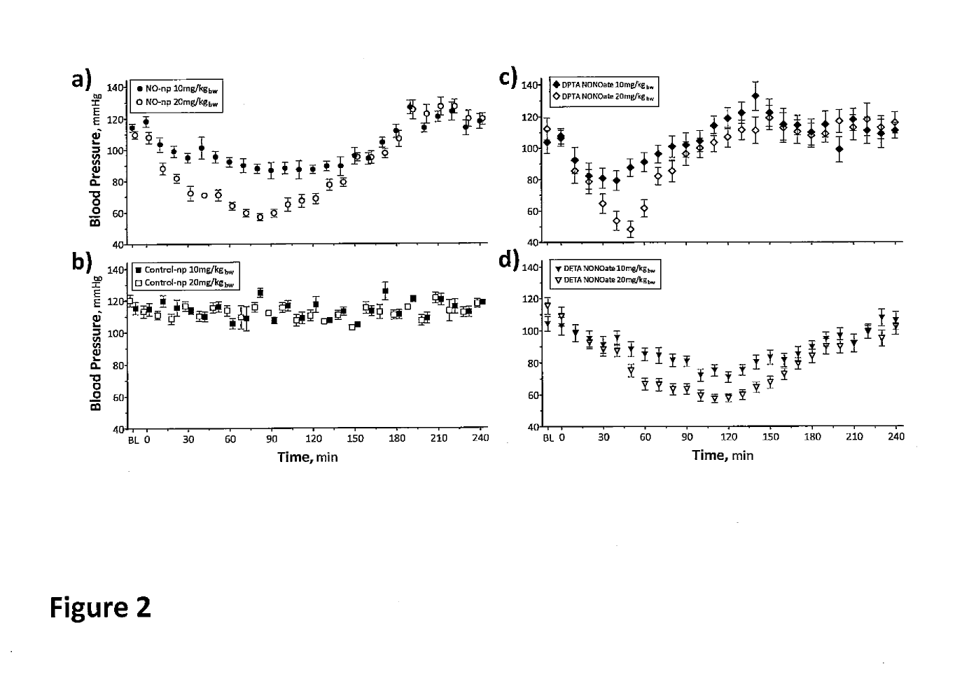

Systemic parameters. MAP decreased after infusion of NO-np in a dose dependent fashion (FIGS. 2A and 213), Maximal decrease in MAP in animals treated with NO-np was measured at 90 min after infusion. Maximal decrease pressure was measured 90 min after infusion. MAP restored to baseline after 3 h after infusion of NO-np. MAP increased above baseline in animals treated with NO-np at 3.5 h after infusion. MetHb levels 4 h after infusion of NO-np were 9±2% for 10 mg/kg and 14±3% for 20 mg/kg, respectively. Infusion of control particles did not change MAP over the 4 h observation period. HR changes after infusion of NO-np were inversely correlated with the changes in MAP, as HR increased above baseline went MAP decreased below baseline.

Infusion of identical amounts of DETA NONOate and DPTA NONOate produced similar decreases in blood pressure (FIGS. 2C and 2D). Importantly though, the impact on vascular tone following NONOate use was highly inefficient as compared to NO-np, with 30 times more NO release required to induce a similar physiologic impact. This is manifested as a significant effect on methemoglobin formation by NONOate administration with subsequent decrease in hemoglobin oxygen carrying capacity. MetHb levels 4 h after infusion of DPTA NONOate were 19±3% for 10 mg/kg and 24±3% for 20 mg/kg, respectively. MetHb levels 4 h after infusion of DETA NONOate were 21±3% for 10 mg/kg and 26±4% for 20 mg/kg, respectively.

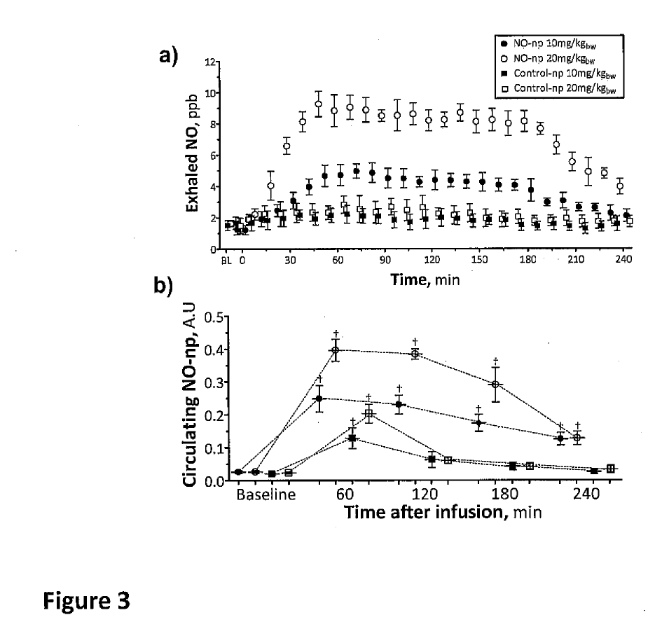

Exhaled. NO after infusion of NO-np. Exhaled NO increased after infusion of NO-np, maximal exhaled NO levels were measured 1 h after infusion (FIG. 3A). Increased exhaled NO levels were maintained over a period of 2 h, after that exhaled NO decreased. Exhaled NO levels in animals treated with Control-np did not increase.

Circulation tune. Intravascular fluorescence is proportional to the concentration of circulating NO-np (FIG. 3B). Infusion of fluorescently label nanoparticles produced an increase in intravascular fluorescence. Circulating particles were observed up 6 hours after infusion, and by next day no evidence of fluorescent labeled NO-np was observed. Circulating labeled NO-np were observed up to 6 h after infusion, but not 24 hours following intravenous introduction. However, control-np were not observed in the circulation 4 hours after infusion.

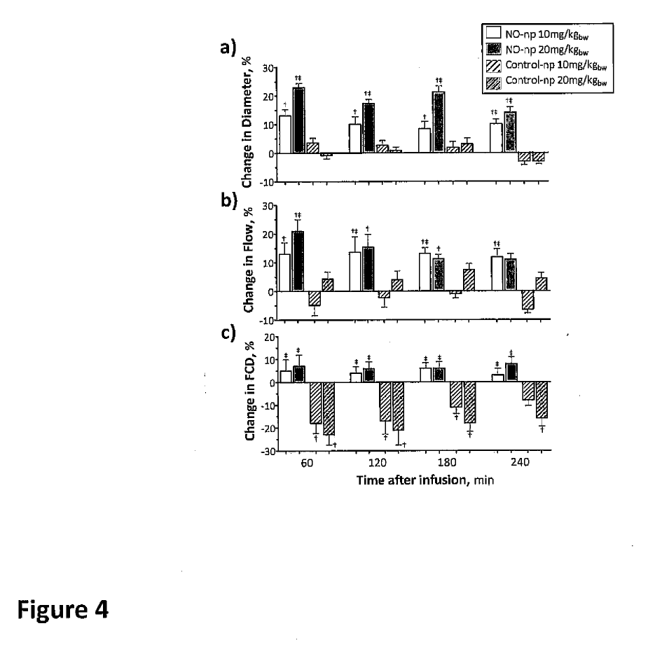

Hemodynamics parameters. Circulating NO-np produced microvascular vasodilatation and increased blood flow compared to baseline and Control-np (FIGS. 4A and 4B). Infusion of control particles did not affect arteriolar diameter or blood flow. In the venular side no significant change in diameter was measured after infusion of NO-np or Control-np. Venular blood flow was reduced from baseline after infusion of 20 mg/kg NO-np. Functional capillary density (FCD) was not affected in animals treated with NO-np, however control nanoparticles reduced the FCD (FIG. 4C).

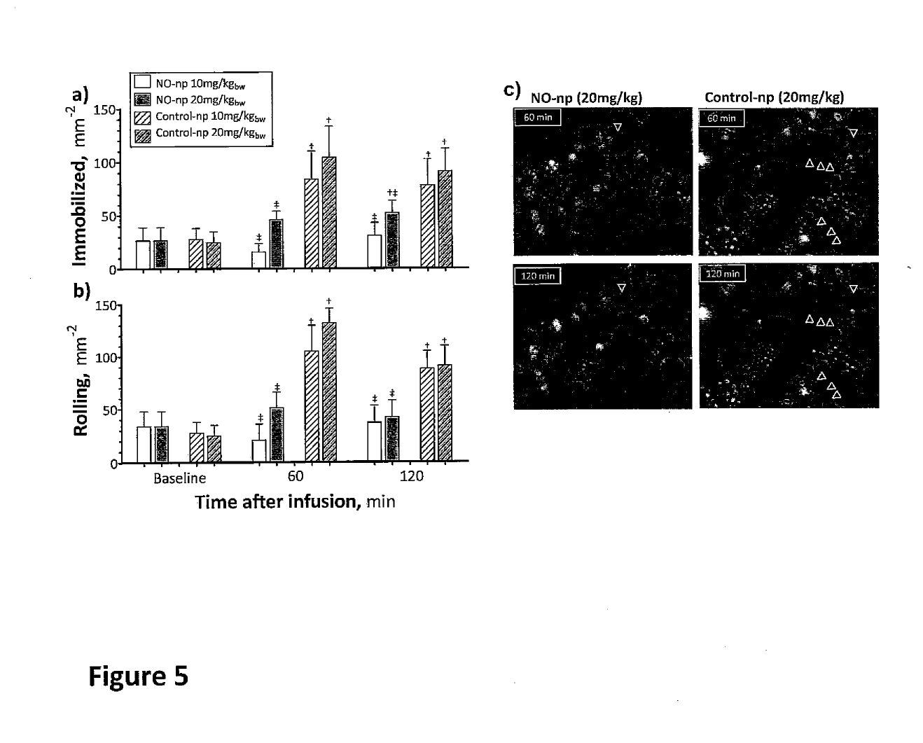

Leukocyte activation parameters. Number of immobilized leukocytes increased after infusion of control particles at 30 min, 1 h and 2 h, respectively (FIGS. 5A, 5B and 5C). NO-np did not increase immobilized leukocytes compared to baseline, although animals treated with 20 mg/kg demonstrated a higher number of immobilized leukocytes as compared to animals treated with 10 mg/kg. Number of rolling leukocytes increased from baseline after infusion of Control-np at all time points. NO-np did not increase the number of rolling leukocytes compared to baseline, and no difference was measured between doses of NO-np.

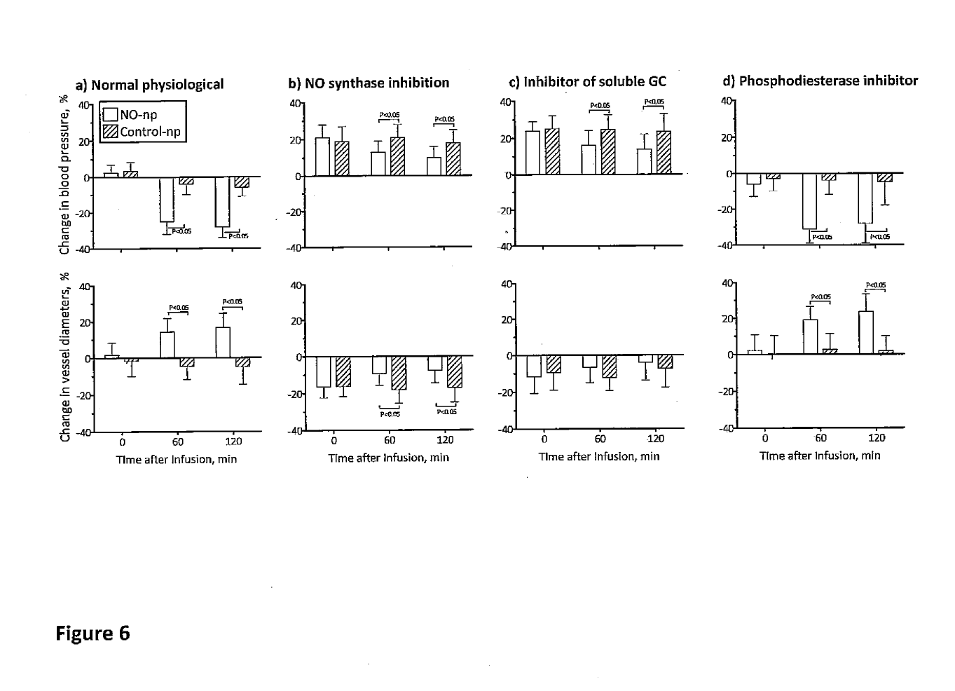

NO synthase and GMP metabolism (10 mg/kg). Infusion of NO-np during NO synthase inhibition with L-NAME, partially reversed the resulting vasoconstriction and increase in MAP as compared to Control-np (FIG. 6B). Infusion of 10 mg/kg of NO-np during irreversible inhibition of heme-site soluble guanylyl cyclase with ODQ resulted in minimal vascular impact, slightly but significantly decreasing the vasoconstriction and hypertension induced by ODQ (FIG. 6C). Infusion of NO-np and Control-np after treatment with the phosphodiesterase inhibitor Zaprinast, increased the vasodilatory effects of the NO-np (FIG. 6D).

### Discussion

The principal finding is that the physiological response to the infusion of NO-np in the circulatory system is consistent with a sustained release of physiologically relevant concentrations of NO. This sustained release is reflected in both the decrease in blood pressure and the increase in vascular relaxation, compared to the absence of any vascular response following the infusion of the Control-np. The efficacy and potential potency of the exogenous NO released by the NO-np is evident from the systemic hypotension and increase in exhaled NO induced by infusion of 20 mg/kg_{bw}. Additionally, the released NO inhibited the intravascular inflammatory response observed with the Control-np, suggesting an immunomodulatory role for this technology. The levels of exhaled NO and modest increase in nitrite levels are not compatible with a scenario in which nitrite is first released and then converted to NO. Furthermore, under the non-acidic physiological conditions there should be minimal NO formation from any released nitrite.

From the physiological viewpoint, exogenous NO released in the intravascular compartment by the NO-np will be partially scavenged by the RBC hemoglobin, and only a portion diffuses abluminally. A minor increase in intra-luminal NO concentration affects the endothelial NO diffusion gradients, thereby allowing the endothelial derived NO to diffuse further from cell to cell, independent of receptors and channels. The consequence of the high reactivity and short half-life of NO is that its biological action and impact are determined primarily by the rate of formation and the diffusion gradients at its site of action. Generation and transport of NO in vivo are closely linked to endothelial cell function, local shear stress, and local oxygen conditions. All of these factors pose a major challenge for systemic NO delivery based on using inhaled gaseous NO delivered from a pressurized tank. This mode of NO delivery which is neither convenient nor cost effective is also potentially harmful given the initial high local concentrations. Conversely, NO-np are stable, easily deployed materials that release pure, biologically relevant non-toxic concentrations of NO in a controllable sustained manner. Pulmonary microcirculation appears to be affected by the NO-np, as the arterial blood pO₂ increased from baseline after infusion of NO-np, as compared to control particles.

Studies with other NO donors indicated that these drugs are generally less potent on intralobar pulmonary arteries (resistance vessels) than on main pulmonary arteries (conduit vessels).[16, 17]

Infusion of NO-np and NONOates decreased blood pressure in a dose dependent fashion, and control-np did not have effect on blood pressure. However, NONOates seem highly inefficient when compared to NO-np, as 30 times more NO release from NONOates was required to produce similar physiologic impact. Therefore, administration of NONOates induced higher methemoglobin formation than NO-np, with subsequent less effect on oxygen carrying capacity. However, a sustained increase in intra-luminal NO affects the endothelial NO diffusion gradients, thus allowing the endothelial derived NO to diffuse further. Reflecting sustained generation/distribution of NO from NO-np, exhaled NO levels increased for several hours after infusion with maximum levels achieved 1 h after infusion. Our microvascular vasodilatation data suggest that the release of NO accounts for vasorelaxant responses produced by the NO-np, as the control nanoparticles did not induce relaxation at corresponding concentrations.

The NO-np are effective vasorelaxants, whose potency is limited by the dose and time course of hydration. Additionally, as a novel class of NO donors, NO-np do not exhibit many of the organic nitrite/nitrate limitations, such as “tolerance,” as well as do not require an external reducing agent. Organic nitrates have been used for over a century to treat angina and the discovery of NO simply served to clarify the mechanism through which these ancient drugs worked.[18] The organic nitrates are the only NO donor used routinely in clinical practice, containing nitroxy ester groups that release NO from a three electron reduction process. Although reducing agents (e.g. thiols) are undoubtedly involved in bioactivation, it is now almost universally assumed that specific enzymes mediate this process.[19, 20] The main limitation of organic nitrates is decreased efficacy with prolonged continuous use; a so-called ‘nitrate tolerance’.[21, 22] The proposed mechanisms for nitrate tolerance include depletion of tissue thiols, de-sensitivity of sGC or increase in breakdown of cGMP by PDEs.[23, 24] NO-np are novel donors without many of the shortcomings of organic nitrates, as NO-np do not require an external reducing agent, and would bypass many of these limitations while retaining and perhaps improving efficacy.

The NO-np mediated vasorelaxation is independent of NOS inhibition, however the NO released by 10 mg/kg_{BW} of NO-np was not sufficient to completely supplant the NO inhibited from the NOS system. The relaxant responses produced by NO-np could not be attributed to nitrite, consistent with earlier in vitro results. [8] The NO-np impact on blood pressure and vessel diameter was diminished by administering ODQ, a selective inhibitor of soluble guanylate cyclase (GC).[25] GC is the enzyme responsible for increasing cyclic guanosine monophosphate (cGMP) levels in response to increasing concentrations of NO and stimulating cGMP-dependent protein kinase (PKG) in the vascular smooth muscle. Inhibition of NO binding to GC acts downstream on the NO action in vasorelaxation, functionally ablating most of the activity of NO on vascular tone. Thus, ODQ-induced mitigation of the previously demonstrated NO-np vascular impact serves to confirm NO as the active species released from the NO-np.

In addition to regulating vascular tone, NO and cGMP signaling can also inhibit proliferation and induce apoptosis in vascular smooth muscle.[26] Intracellular cGMP levels are determined by the rate of cGMP hydrolysis as well as synthesis. The major enzymes responsible for degrading cGMP in vascular smooth muscle are cGMP-specific phosphodiesterase (PDE) Type 5 (PDE5) and calcium/calmodulin-dependent PDE Type 1.[15] Infusion of NO-np in animals pretreated with PDE5 inhibitor Zaprinast increased the effects of NO-np on blood pressure and more so on vessel diameter. Thus, acute inhibition of PDE5 activity, by drugs such as sildenafil, combined with NO-np represents an attractive therapeutic strategy to manipulate systemic vascular tone.

There are strong indications that activated macrophages can recognize nanoparticles in the blood.[27] The presence of exogenous NO from the nanoparticles prevented inflammatory reaction to external agents, especially the number of immobilized and rolling leukocytes. As not all macrophages and monocytes are identical[28], macrophages found within microcirculatory regions are more active in cytokine production and have stronger phagocytic receptors.[29] NO-np technology could prevent long-circulating nanoparticles from being trapped by such macrophage populations and this action may be even further enhanced with human macrophages. The NO-np technology seems to be optimal for “macrophage-evasion,” providing a long-circulating drug reservoir from which the drug can be released into the vascular compartment in a continuous and controlled manner.

Here we present the physiological responses to intravascular infusion of NO-np, consistent with a sustained release of physiologically relevant levels of NO, reflected by decreased blood pressure and increased vascular relaxation. The efficacy and potency of the exogenous NO released by the NO-np is evident from the systemic hypotension and increase in exhaled concentrations of NO. Additionally, NO-np do not exhibit many of the shortcomings of current methods of applying NO in clinical settings, suggesting that it may be a conduit through which the potential of NO can be harnessed and translated into a practical therapeutic strategy. In summation, the presented results indicate that the NO-np formulations has considerable promise both for therapeutic applications requiring sustained systemic NO delivery, and for basic research focusing on the behavior and activity of intravascular NO.

### REFERENCES FOR EXAMPLE 1

[1] Zacharia, I. G.; Deen, W. M. Diffusivity and solubility of nitric oxide in water and saline. Ann Biomed Eng 33:214-222; 2005.
[2] Moncada, S.; Palmer, M. J.; Higgs, E. A. Nitric Oxide: Physiology, pathophysiology, and pharmacology. Pharmacol Rev 43:109-134; 1991.
[3] Busse, R.; Fleming, I. Pulsatile stretch and shear stress: physical stimuli determining the production of endothelium-derived relaxing factors. J Vasc Res 35:73-84; 1998.
[4] Lancaster, J. R., Jr. A tutorial on the diffusibility and reactivity of free nitric oxide. Nitric Oxide 1:18-30; 1997.
[5] Ichinose, F.; Roberts, J. D., Jr.; Zapol, W. M. Inhaled nitric oxide: a selective pulmonary vasodilator: current uses and therapeutic potential. Circulation 109:3106-3111; 2004.
[6] Zapol, W. M. Inhaled nitric oxide. Acta Anaesthesiol Scand Suppl 109:81-83; 1996.
[7] Homer, K.; Wanstall, J. In vitro comparison of two NONOates (novel nitric oxide donors) on rat pulmonary arteries. Eur J Pharmacol 356:49-57; 1998.
[8] Friedman, A. J.; Han, G.; Navati, M. S.; Chacko, M.; Gunther, L.; Alfieri, A.; Friedman, J. M. Sustained release nitric oxide releasing nanoparticles: characterization of a novel delivery platform based on nitrite containing hydrogel/glass composites. Nitric Oxide 19:12-20; 2008.
[9] Maragos, C. M.; Morley, D.; Wink, D. A.; Dunams, T. M.; Saavedra, J. R; Hoffman, A.; Bove, A. A.; Isaac, L.; Hrabie, J. A.; Keefer, L. K. Complexes of .NO with nucleophiles as agents for the controlled biological release of nitric oxide. Vasorelaxant effects. J Med Chem 34:3242-3247; 1991.
[10] Maragos, C. M.; Wang, J. M.; Hrabie, J. A.; Oppenheim, J. J.; Keefer, L. K. Nitric oxide/nucleophile complexes inhibit the in vitro proliferation of A375 melanoma cells via nitric oxide release. Cancer Res 53:564-568; 1993.
[11] Homer, K. L.; Wanstall, J. C. Cyclic GMP-independent relaxation of rat pulmonary artery by spermine NONOate, a diazeniumdiolate nitric oxide donor. Br J Pharmacol 131:673-682; 2000.
[12] Endrich, B.; Asaishi, K.; Götz, A.; Messmer, K. Technical report: A new chamber technique for microvascular studies in unanaesthetized hamsters. Res Exp Med 177:125-134; 1980.
[13] Childs, E. W.; Udobi, K. F.; Wood, J. G.; Hunter, F. A.; Smalley, D. M.; Cheung, L. Y. In vivo visualization of reactive oxidants and leukocyte-endothelial adherence following hemorrhagic shock. Shock 18:423-427; 2002.
[14] Schrammel, A.; Behrends, S.; Schmidt, K.; Koesling, D.; Mayer, B. Characterization of 1H-[1,2,4]oxadiazolo[4,3-a]quinoxalin-1-one as a heme-site inhibitor so of nitric oxide-sensitive guanylyl cyclase. Mol Pharmacol 50:1-5; 1996.
[15] Rybalkin, S. D.; Yan, C.; Bornfeldt, K. E.; Beavo, J. A. Cyclic GMP phosphodiesterases and regulation of smooth muscle function. Circ Res 93:280-291; 2003.
[16] Allman, K. G.; Young, J. D.; Carapiet, D.; Stevens, J. E.; Ostman-Smith, I.; Archer, L. N. Effects of oxygen and nitric oxide in oxygen on pulmonary arterial pressures of children with congenital cardiac defects. Pediatr Cardiol 17:246-250; 1996.
[17] Wanstall, J. C.; Kaye, J. A.; Gambino, A. The in vitro pulmonary vascular effects of FK409 (nitric oxide donor): a study in normotensive and pulmonary hypertensive rats. Br J Pharmacol 121:280-286; 1997.
[18] Bauer, J. A.; Booth, B. P.; Fung, H. L. Nitric oxide donors: biochemical pharmacology and therapeutics. Adv Pharmacol 34:361-381; 1995.
[19] Bennett, B. M.; McDonald, B. J.; Nigam, R.; Simon, W. C. Biotransformation of organic nitrates and vascular smooth muscle cell function. Trends Pharmacol Sci 15:245-249; 1994.
[20] McGuire, J. J.; Anderson, D. J.; Bennett, B. M. Inhibition of the biotransformation and pharmacological actions of glyceryl trinitrate by the flavoprotein inhibitor, diphenyleneiodonium sulfate. J Pharmacol Exp Ther 271:708-714; 1994.
[21] Gori, T.; Parker, J. D. The puzzle of nitrate tolerance: pieces smaller than we thought? Circulation 106:2404-2408; 2002.
[22] Gori, T.; Parker, J. D. Nitrate tolerance: a unifying hypothesis. Circulation 106:2510-2513; 2002.
[23] Axelsson, K. L.; Andersson, R. G. Tolerance towards nitroglycerin, induced in vivo, is correlated to a reduced cGMP response and an alteration in cGMP turnover. Eur J Pharmacol 88:71-79; 1983.
[24] Kim, D.; Rybalkin, S. D.; Pi, X.; Wang, Y.; Zhang, C.; Munzel, T.; Beavo, J. A.; Berk, B. C.; Yan, C. Upregulation of phosphodiesterase 1A1 expression is associated with the development of nitrate tolerance. Circulation 104:2338-2343; 2001.
[25] Garthwaite, J.; Southam, E.; Boulton, C. L.; Nielsen, E. B.; Schmidt, K.; Mayer, B. Potent and selective inhibition of nitric oxide-sensitive guanylyl cyclase by 1H-[1,2,4] oxadiazolo [4,3-a]quinoxalin-1-one. Mol Pharmacol 48:184-188; 1995.
[26] Pollman, M. J.; Yamada, T.; Horiuchi, M.; Gibbons, G. H. Vasoactive substances regulate vascular smooth muscle cell apoptosis. Countervailing influences of nitric oxide and angiotensin II. Circ Res 79:748-756; 1996.
[27] Moghimi, S. M.; Gray, T. A single dose of intravenously injected poloxamine-coated long-circulating particles triggers macrophage clearance of subsequent doses in rats. Clin Sci (Lond) 93:371-379; 1997.
[28] Rutherford, M. S.; Witsell, A.; Schook, L. B. Mechanisms generating functionally heterogeneous macrophages: chaos revisited. J Leukoc Biol 53:602-618; 1993.
[29] Ito, S.; Naito, M.; Kobayashi, Y.; Takatsuka, H.; Jiang, S.; Usuda, H.; Umezu, H.; Hasegawa, G.; Arakawa, M.; Shultz, L. D.; Elomaa, O.; Tryggvason, K. Roles of a macrophage receptor with collagenous structure (MARCO) in host defense and heterogeneity of splenic marginal zone macrophages. Arch Histol Cytol 62:83-95; 1999.
[30] Martinez, L. R.; Han, G.; Chacko, M.; Mihu, M. R.; Jacobson, M.; Gialanella, P.; Friedman, A. J.; Nosanchuk, J. D.; Friedman, J. M. Antimicrobial and Healing Efficacy is of Sustained Release Nitric Oxide Nanoparticles Against Staphylococcus Aureus Skin Infection. J Invest Dermatol; 2009.

### Example 2

Reverting Vasoconstriction of Polymerized Hemoglobin with Sustained Release of Nitric Oxide Form Long-Circulating Nanoparticles

In this study, the central objective was to establish the efficacy of NO releasing nanoparticles (NO-np) to restore smooth muscle NO bioavailability, to revert vasoconstriction and hypertension induced by NO scavenging of polymerized bovine Hb (PBH, Oxyglobin™, OPK Biothech, Cambridge, Mass.; approved in the United States and European Union for veterinary use). Thus, the purpose of this study was to examine the potential benefit of combining NO-np (NO releasing nanoparticles) with glutaraldehyde-polymerized bovine Hb infusion into conscious hamster to determine the potential hemodynamic improvements due to NO-np co-adjunct therapy. It was hypothesized that NO-np would counter both the systemic hypertension and decrease vasoconstrictor effects of PBH infusion, improving systemic and microvascular function. The study was performed using the hamster window chamber model to asses concurrently systemic and microvascular parameters. PBH was infused at stock concentration (13 g/dl) in a volume equal to 10% of the animal estimated blood volume (7% body weight), and intravascular NO supplementation was provided with NO-np (10 and 20 mg/kg of body weight). Nanoparticles free of NO were used as control. In vivo experimental evidence that intravascular exogenous supplementation of NO using NO-np prevented cardiovascular complication is induced by acellular Hb scavenging of NO. Given the critical importance of NO in regulating pathophysiologic states involving the vasculature, controlled and sustained intravascular NO delivery could profoundly impact current treatment of hemolytic, metabolic and endothelial dysfunction induced vascular disease states.

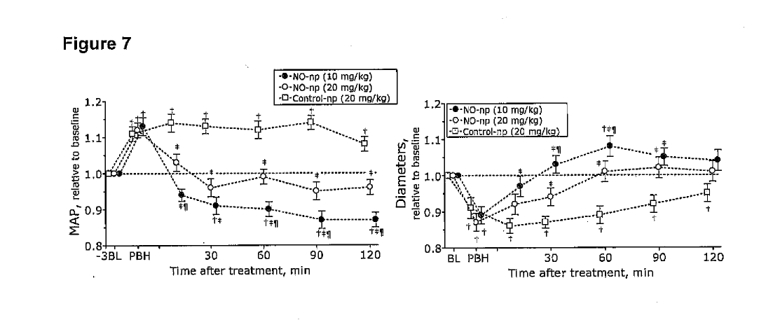

PBH infusion. An equivalent volume to 10% of the animal blood volume (calculated as 7% of the animal body weight) of polymerized bovine Hb (PBH, 13 g/dl, Oxyglobin™, is OPK Biothech, Cambridge, Mass.) was infused via the jugular vein at a rate of 0.1 ml/min (top load). FIG. 7 presents the experimental protocol.

NO releasing nanoparticles infusion. NO releasing nanoparticles were infused 10 min after PBH infusion. They were suspended in deoxygenated saline, and infused in a volume of 50 μl (equivalent to less than 2% of the animals blood volume) via the jugular vein at a rate lower than 100 μl/min.

Experimental groups. After infusion of PBH, animals were randomly divided into three experimental groups. Groups are named based on the type of nanoparticles used and concentration infused, namely: NO-np (10 mg/kg), therapy of 10 mg/kg of NO releasing nanoparticles; NO-np (20 mg/kg), therapy of 20 mg/kg of NO releasing nanoparticles; and Control-np (20 mg/kg), therapy of 20 mg/kg of NO free nanoparticles. Only one concentration of Control-np (20 mg/kg) was used, as there is not expected effect in NO bioavailability from this particles in circulation.

### Results

Blood pressure and heart rate. Infusion of PBH increased MAP in all groups, absolute baseline values are presented in FIG. 7. Treatment with 10 mg/kg of NO-np decreased MAP compare to Control-np, and the higher dose of NO-np (20 mg/kg) decreased MAP compare to baseline, and treatments with Control-np. The decrease of PBH-induced hypertension with the NO-np extended the 2 hours observation. Treatment with 20 mg/kg of NO-np increased HR compared to Control-np, the low dose of NO-np (10 mg/kg) did not have any effects in HR.

Arteriolar vessel diameter and perfusion. Infusion of PBH produced significant vasoconstriction in all groups and only treatment with NO-np was able to restore arteriolar diameter. Diameters of groups treated with Control-np, remained constricted over 2 hours. Arteriolar flows decreased from baseline after infusion of PBH, NO-np reverted decrease in arteriolar blood flow. Treatment with NO-np has significantly higher flows compare to Control-np.

The main result of our experiments was that the observed increases in systemic blood pressure and peripheral vasoconstriction associated with PBH in circulation can be prevented by co-administration of NO releasing nanoparticles (NO-np), to restore NO levels, and therefore reduce the associated decline in blood blow and oxygen delivery. Infusion of NO-np in situations where vascular NO bioavailability is decreased, such as acellular Hb or hemolysys, can prevent NO scavenging-induced vascular complications, by a consistent and sustained release of NO from NO-np to reinstate the NO consumed by NO dioxygenase reaction.

### Example 3

### Methods

Synthesis of NO-nps and Control-nps. A hydrogel/glass composite was synthesized using a mixture of tetramethylorthosilicate, polyethylene glycol, chitosan, glucose, and sodium nitrite in a 0.5 M sodium phosphate buffer (pH 7)¹². As previously described, nitrite is reduced to NO within the matrix as a result of the glasys properties of the composite, which allow for redox reactions initiated, with thermally generated electrons from glucose¹². The composite is dried using a lyophilizer and crushed using planetary ball mill, resulting in nanoparticles containing NO. Once exposed to an aqueous environment, the hydrogel allows for an opening of the water channels inside the particles, facilitating the release of the trapped NO over extended time periods. Control-nps, free of NO inside, were produced identically, but without nitrite.

Animal Preparation. Investigations were performed in 50-65 g male Golden Syrian Hamsters (Charles River Laboratories, Boston, Mass.) fitted with a dorsal skinfold chamber window. Animal handling and care followed the NIH Guide for the Care and Use of Laboratory Animals. The experimental protocol was approved by the local animal care committee. The hamster chamber window model is widely used for microvascular studies in the unanesthetized state, and the complete surgical technique is described in detail elsewhere¹⁴. Catheters were tunneled under the skin and exteriorized at the dorsal side of the neck, and securely attached to the window frame.

Inclusion Criteria. Animals were suitable for the experiments if: 1) systemic parameters were within normal range, namely, heart rate (HR)>340 beat/min, mean arterial blood pressure (MAP)>80 mmHg, systemic Hct >45%, and arterial oxygen partial pressure (PaO₂)>50 mmHg; and 2) microscopic examination of the tissue in the chamber observed under a ×650 magnification did not reveal signs of edema or bleeding.

Acute hemorrhage protocol. Acute hemorrhage was induced by withdrawing 50% of estimated total blood volume (BV) via the carotid artery catheter within 5 min. Total BV was estimated as 7% of body weight. Animals were followed over 90 min after hemorrhage induction. The animals were categorized as non-survivors and euthanized earlier if at any time during the protocol their MAP fell below 30 mmHg for more than 10 minutes.

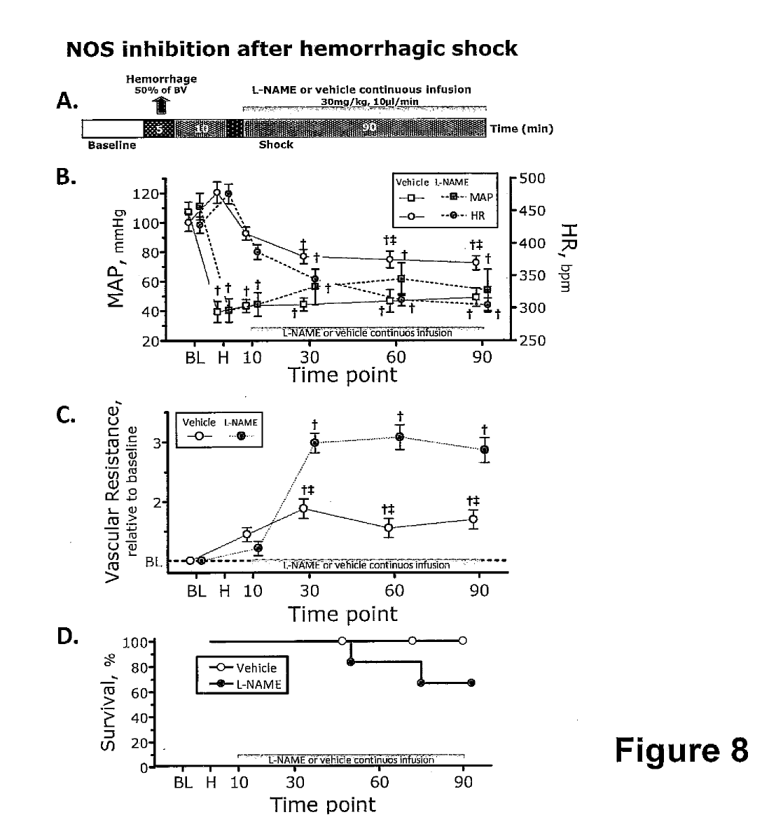

NO synthase during hemorrhagic shock NOS inhibition was induced by continuous infusion of L-NAME (N-nitro-L-arginine methyl ester, 30 mg/kg, 10 μl/min; Sigma-Aldrich. St. Louis, Mo.). Vehicle groups received saline at identical rate. L-NAME and vehicle infusion started 10 min after the end of the hemorrhage. FIG. 8A presents the experimental protocol.

Experimental groups for NO supplementation. Nanoparticles were infused 10 min after the hemorrhage, suspended in 50 μl deoxygenated saline, and infused via the jugular vein (100 μl/min). Post-hemorrhage, animals were randomly divided¹⁵ into three groups: 1) NO-nps, 5 mg/kg of NO-nps; 2) Control-nps, 5 mg/kg of NO free nanoparticles; 3) Vehicle, 50 μl deoxygenated saline. Eighteen (n=18) animals were entered into the study, and assigned to the following experimental groups: NO-lips (n=6); Control-nps (n=6); and Vehicle (n=6).

Systemic Parameters. MAP and heart rate (HR) were recorded continuously (MP 150, Biopac System; Santa Barbara, Calif.). Hct was measured from centrifuged arterial blood samples taken in heparinized capillary tubes. Hb content was determined spectrophotometrically from a single drop of blood (Hemocue, Stockholm, Sweden).

Blood Chemistry and Biophysical Properties. Arterial blood was collected in heparinized glass capillaries (0.05 ml) and immediately analyzed for PaO₂, PaCO₂ and pH (Blood Chemistry Analyzer 248, Bayer, Norwood, Mass.). The comparatively low PaO₂ and high PaCO₂ of these animals is a consequence of their adaptation to a fossorial environment.

Methemoglobin Measurement. Methemoglobin was established according to Winterbourn¹⁶. Calibration is ensured using standard levels at 5.2%, 2.6% and 1.2% MetHb (RNA Medical, Bayer Diagnostics Medfield, Mass.).

Plasma nitrite/nitrate. Blood samples were collected from carotid artery, and centrifuged to separate RBCs and plasma. Plasma proteins were removed by adding equal volume of methanol, and centrifuged at 15000 rpm for 10 min. Concentrations of NOx in the supernatant were measured with a NOx analyzer (ENO-20; Eicom, Kyoto, Japan). This analyzer combines Griess method and high-performance liquid chromatography.

Microvascular Experimental Setup. The unanesthetized animal was placed in a restraining tube with a longitudinal slit from which the window chamber protruded, then fixed to the microscopic stage of a transillumination intravital microscope (BX51WI, Olympus, New Hyde Park, N.Y.). The animals were given 20 min to adjust to the change in the tube environment before measurements. The tissue image was projected onto a charge-coupled device camera (COHU 4815) connected to a videocassette recorder and viewed on a monitor. Measurements were carried out using a 40× (LUMPFL-WIR, numerical aperture 0.8, Olympus) water immersion objective. The same sites of study were followed throughout the experiment so that comparisons could be made directly to baseline levels.

Microhemodynamics. Arteriolar and venular blood flow velocities were measured on-line by using the photodiode cross-correlation method (Velocity Tracker Model 102B, Vista Electronics, San Diego, Calif.)¹⁷. Measured centerline velocity was corrected according to vessel size to obtain mean RBC velocity (V)¹⁸. Video image-shearing method was used to measure vessel diameter (D)¹⁹. Blood flow (Q) was calculated from the measured values as Q=π×V (D/2)². This calculation assumes a parabolic velocity profile and has been found to be applicable to tubes of 15-80 μm internal diameters and for Hcts in the range of 6-60%¹⁸. Changes in arteriolar and venular diameter from so baseline were used as indicators of a change in vascular tone. Peripheral vascular resistance (PVR) was calculated in terms of pressure (MAP) divide by local blood flow (Q), by mathematically using the Hagen-Poiseuille equation. PVR relative to baseline was calculated by ratio of MAP and Q normalized to baseline. Peripheral vascular hindrance was calculated by normalizing PVR by blood viscosity normalized to baseline, to eliminate viscous resistance component from the Poiseuille equation. Wall shear stress (WSS) was defined by WSS=WSR×η, where WSR is the wall shear rate given by 8V×D⁻¹, and η is blood viscosity.

Functional Capillary Density (FCD). Functional capillaries, defined as those capillary segments that have RBC transit of at least one RBC in a 45 s period in 10 successive microscopic fields were assessed, totaling a region of 0.46 mm². FCD (cm⁻¹), i.e., total length of RBC perfused capillaries divided by the area of the microscopic field of view, was evaluated by measuring and adding the length of capillaries that had RBC transit in the field of view. Changes in FCD from baseline were used as indictors of capillary perfusion.

Data analysis. Results are presented as mean standard deviation. Data within each group were analyzed using analysis of variance for repeated measurements (ANOVA, Kruskal-Wallis test). When appropriate, post hoc analyses were performed with the Dunns multiple comparison test. Data between groups was analyzed using two-way ANOVA nonparametric repeated measurements, and, when appropriate, post hoc analyses were performed using Bonferroni tests. The Grubbs' method was used to assess closeness for all measured parameters values at baseline. Microhemodynamic measurements were compared to baseline levels obtained before the experimental procedure, for more robust statistics for small sample populations. All statistics were calculated using GraphPad Prism 4.01 (GraphPad Software, Inc., San Diego, Calif.). Changes were considered statistically significant if P<0.05.

### Results

NOS inhibition during hemorrhagic shock. Twelve hamsters were used to establish effects of NO inhibition (L-NAME: n=6; 68.4±8.2 g; and Vehicle: n=6; 66.2±7.1 g).

Hemorrhage significantly decreased Hct and Hb in both groups. Changes in MAP and HR post hemorrhage are shown in FIG. 8B. MAP decreased after hemorrhage, and NOS inhibition produced an initial recovery in pressure, however some animals were not able to maintain the pressure. HR initially increased after hemorrhage, and NOS inhibition reduced HR compared to baseline and Vehicle. Diameters were constricted after hemorrhage, and NOS inhibition increased vasoconstriction compared to baseline and Vehicle. Microvascular flows and FCD were significantly decreased after hemorrhage compared to baseline in both groups. Peripheral vascular resistance is presented in FIG. 8C. Two animals treated with L-NAME did not survive the experimental protocol (47 and 72 minutes), FIG. 8D.

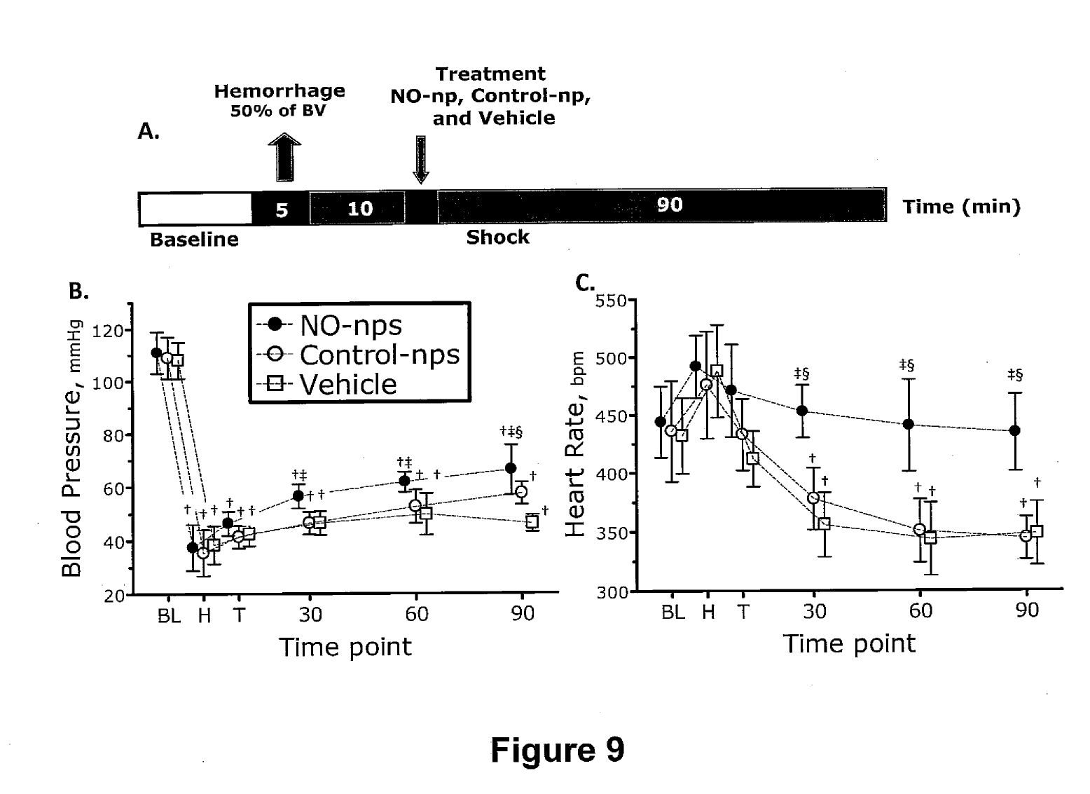

NO supplementation during hemorrhagic shock. A second group of animals was used to study the role of NO supplementation during hemorrhagic shock, divided in three groups: NO-nps: (n=6; 66.2±5.1 g); Control-nps: (n=6; 63.8±4.4 g); and Vehicle: (n=6; 66.1±4.8 g). FIG. 9A presents the experimental protocol. All animals tolerated the experimental protocol. All animals passed Grubbs' test ensuring that parameter at baseline were within a similar population.

Blood gas chemistry. Blood chemistry results are presented in Table 2. Hemorrhage decreased Hct and Hb in all groups. MetHb increased in animals treated with NO-nps compared to Control-nps and Vehicle. Plasma nitrite and nitrate decreased from baseline in animals that received the Vehicle or Control-nps, while animals treated with NO-nps presented and increased compared to baseline and to animals treated with Vehicle and Control-nps. Arterial blood pO₂ after hemorrhage increased in all groups compared to baseline. However, animals treated with NO-nps had lower arterial pO₂ compared to Vehicle and Control-nps. Arterial blood pCO₂ after hemorrhage decreased in all groups compared to baseline, although, animals treated with NO-nps had higher arterial pCO₂ compared to Vehicle and Control-nps. Arterial blood pH after hemorrhage decreased in all groups compared to baseline, and animals treated with NO-nps demonstrated a higher arterial pH as compared to Vehicle and Control-np. Blood and plasma viscosity were decreased compared to baseline in all groups. Plasma colloidal osmotic pressure (COP) decreased from baseline in all groups.

TABLE 2
Systemic parameters

| | | Hemorrhagic Shock | | | | | |
|---|---|---|---|---|---|---|---|
| | | 60 min | | | 90 min | | |
| | Baseline | Vehicle | Control-nps | NO-nps | Vehicle | Control-nps | NO-nps |
| n | 18 | 6 | 6 | 6 | 6 | 6 | 6 |
| Hct, % | 49 ± 1 | 29 ± 1^{†} | 30 ± 2^{†} | 29 ± 2^{†} | 27 ± 1^{†} | 28 ± 1^{†} | 27 ± 1^{†} |
| Total Hb, g/dl | 14.7 ± 0.5 | 9.2 ± 0.6^{†} | 9.4 ± 0.7^{†} | 9.3 ± 0.6^{†} | 8.7 ± 0.4^{†} | 9.0 ± 0.6^{†} | 8.9 ± 0.5^{†} |
| MetHb, % | — | — | — | 4 ± 2 | — | — | 6 ± 2 |
| Plasma | | | | | | | |
| NO₂⁻, ηM | 477 ± 64 | 346 ± 62^{†} | 402 ± 63^{†} | 721 ± 126^{†‡} | 369 ± 64^{†} | 345 ± 54^{†} | 818 ± 153^{†‡} |
| NO₃⁻, μM | 1.5 ± 0.3 | 1.0 ± 0.2^{†} | 0.9 ± 0.3^{†} | 2.1 ± 0.8^{†‡} | 0.8 ± 0.2^{†} | 0.9 ± 0.2^{†} | 2.4 ± 0.7^{†‡} |
| pₐO₂, mmHg | 58.2 ± 6.3 | 100.6 ± 7.0^{†} | 98.7 ± 6.8^{†} | 71.7 ± 6.5^{†‡} | 97.4 ± 6.8^{†} | 97.0 ± 7.2^{†} | 69.2 ± 6.1^{†‡} |
| pₐCO₂, mmHg | 53.2 ± 4.7 | 35.4 ± 6.3^{†} | 37.6 ± 6.1^{†} | 44.5 ± 6.8^{†‡} | 36.1 ± 6.0^{†} | 35.6 ± 5.8^{†} | 45.5 ± 6.0^{†‡} |
| pHₐ | 7.337 ± 0.022 | 7.230 ± 0.029^{†} | 7.231 ± 0.033^{†} | 7.331 ± 0.026^{†‡} | 7.328 ± 0.024^{†} | 7.327 ± 0.032^{†} | 7.330 ± 0.025^{†‡} |
| BEₐ, mmol | 2.9 ± 1.3 | −3.2 ± 1.8^{†} | −2.9 ± 1.5^{†} | 0.2 ± 1.0^{†‡} | −3.4 ± 1.6^{†} | −3.5 ± 1.8^{†} | 0.3 ± 1.4^{†‡} |
| Viscosity | | | | | | | |
| Blood*, cP | 4.2 ± 0.2 | | | | 3.1 ± 0.4^{†} | 3.2 ± 0.5^{†} | 3.0 ± 0.3^{†} |
| Plasma*, cP | 1.2 ± 0.1 | | | | 0.9 ± 0.2^{†} | 1.0 ± 0.2^{†} | 1.0 ± 0.2^{†} |
| COP, mmHg | 17.8 ± 2.3 | | | | 13.8 ± 2.4^{†} | 13.6 ± 1.8^{†} | 13.9 ± 1.7^{†} |

Values are means ± SD. Baseline included all animals. No significant differences were detected between baseline. Hct, hematocrit; Hb, hemoglobin; NO₂⁻, nitrite; NO₃⁻, nitrate; PₐO₂, arterial partial O₂ pressure; PₐCO₂, arterial partial pressure of CO₂; BEₐ, arterial base excess; COP, colloidal osmotic pressure.
^{†}P < 0.05 compared to Baseline;
^{‡}P < 0.05 compared to Vehicle;
§P < 0.05 compared to Control-np.

Changes in MAP and HR post hemorrhage for all groups are shown in FIGS. 9B and 9C. MAP in all groups decreased compared to baseline. Animals treated with NO-nps recovered MAP compared to vehicle within 30 min from the treatment, and after 90 min compared to Control-np. HR in all groups increased after hemorrhage, and after treatment HR dropped in animals treated with Control-nps and Vehicle. However, animals treated with NO-nps maintained HR over the observation time, and higher compared to animals treated with Control-nps and Vehicle.

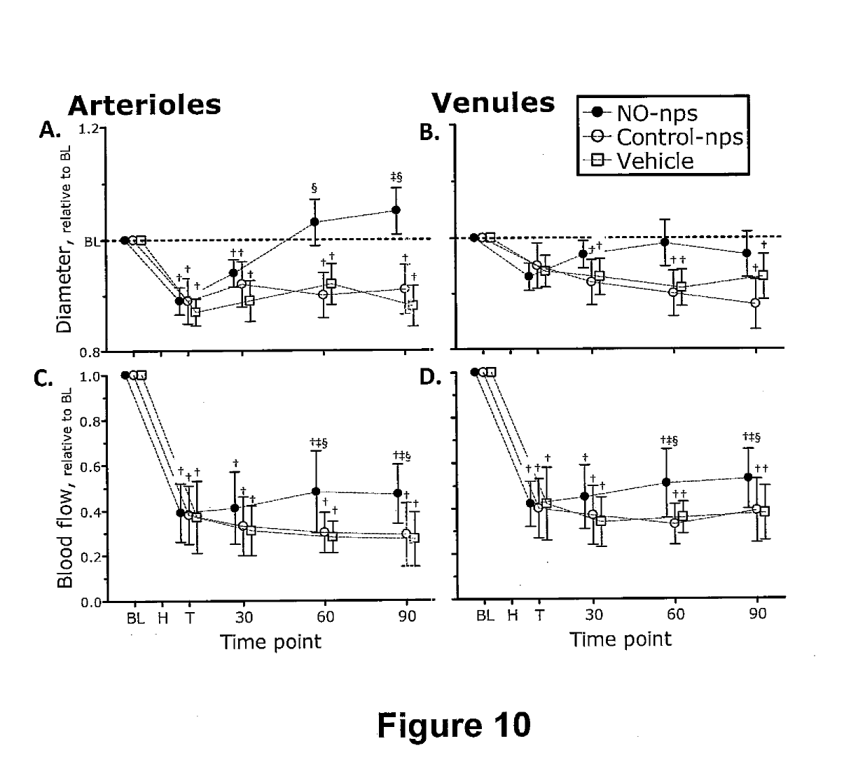

Microvascular diameters and both arteriolar and venular blood flow are presented in FIG. 10. Arteriolar and venular diameters (FIGS. 10A and 10B) were significantly constricted from baseline after hemorrhage for all groups. Microvascular flows were significantly decreased after hemorrhage compared to baseline for all groups. Arteriolar diameters remained constricted compared to baseline for Control-nps and Vehicle treated groups. Conversely, animals treated with NO-nps restored arteriolar diameters, and significant dilation was witnessed, compared to Control-nps and Vehicle treated groups. Arteriolar microvascular blood flow (FIGS. 10C and 10D) for all groups remained lower than baseline, although animals treated with NO-nps demonstrated higher arteriolar flow as compared to Control-nps and Vehicle treated animals. Venular diameters were constricted after hemorrhage for all groups compared to baseline, and remained constricted for Control-nps and Vehicle treated groups. Venular diameters in animals treated with NO-nps were not different from baseline. Venular blood flow decreased after hemorrhage compared to baseline, and remained lower than baseline for all groups. NO-nps had significantly higher venular blood flow as compared to Control-nps and Vehicle.

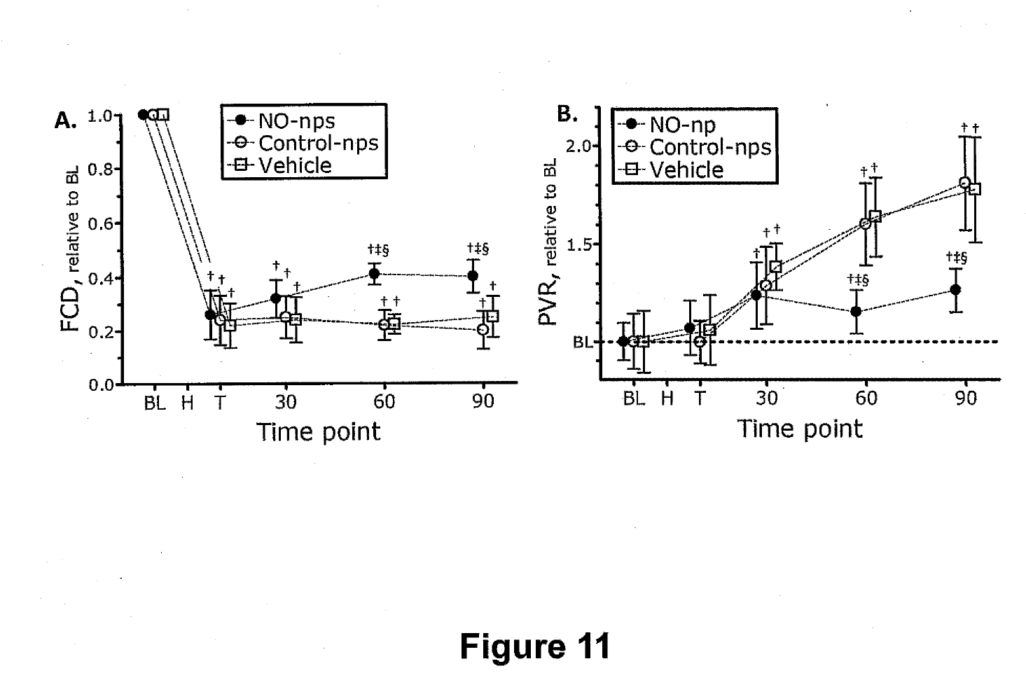

Changes in capillary perfusion during the protocol are presented in FIG. 11A. FCD was significantly reduced after hemorrhage for all groups, at all time points. FCD partially recovered in animals treated with NO-nps compared to Control-nps and Vehicle treated animals.

Estimated PVR are presented in FIG. 11B. PVR increased over the observation time for animals treated with Control-nps and Vehicle, significantly higher when compared to the NO-nps treated group. Calculated hemodynamic parameters are presented in Table 3, Peripheral vascular hindrance, which reflects the contribution of vascular geometry, was a increased compared to baseline after hemorrhage independent of the treatment. Arteriolar and venular vessel WSR and WSS decreased after hemorrhage, compared to baseline, and remained decreased, independent of the treatment.

TABLE 3
Estimated vascular resistance and wall shear rate and

| | | Hemorrhagic Shock 90 min | | |
|---|---|---|---|---|
| | Baseline | Vehicle | Control-nps | NO-nps |
| Vascular | 1.0 | 1.8 ± 0.3^{†} | 1.7 ± 0.4^{†} | 1.3 ± 0.3^{†} |
| resistance, | | | | |
| relative to | | | | |
| baseline | | | | |
| Vascular | 1.0 | 2.5 ± 0.5^{†} | 2.5 ± 0.6^{†} | 1.7 ± 0.5^{†} |
| hindrance | | | | |
| relative to | | | | |
| baseline | | | | |
| Arteriolar | | | | |
| WSR, s⁻¹ | 706 ± 92 | 258 ± 57^{†} | 254 ± 52^{†} | 292 ± 64^{†} |
| WSS, dyn · cm⁻² | 30 ± 4 | 8 ± 2^{†} | 8 ± 2^{†} | 9 ± 2^{†} |
| Venular | | | | |
| WSR, s⁻¹ | 456 ± 84 | 196 ± 48^{†} | 244 ± 51^{†} | 268 ± 46^{†} |
| WSS, dyn · cm⁻² | 20 ± 4 | 7 ± 2^{†} | 8 ± 2^{†} | 8 ± 2^{†} |

Hematocrits are presented in Table 1.
WSR, wall shear rate;
WSS, wall shear stress.
^{†}P < 0.05 compared to Baseline;
‡, P < 0.05 compared to Vehicle;
§, P < 0.05 compared to Control-np.

### Discussion

The principal finding of the study is that treatment of hemorrhagic shock by NO supplementation via NO-nps prevented cardiovascular collapse, and allowed the animals to maintain superior systemic and microvascular hemodynamic conditions as compared to controls. The importance of providing sustained exogenous NO after hemorrhage was evidenced in this study in the form of maintenance of heart rate, microvascular function, FCD and vascular resistance. Our findings relate to scenarios where an increase in NO bioavailability could potentially alter the course and ultimately survival, until volume resuscitation is available. Our results support the hypothesis that severe hemorrhage induces vascular decompensation associated with low NO availability, and that NO supplementation prevents the risks of profound hemorrhage. As generation of NO in vivo is closely linked to endothelial cell function, local shear stress, and local oxygen conditions, the lack of differences in WSS and Hct among groups, suggests that the differences observed between groups are mostly due to the NO released from NO-np.

Hemorrhagic hypotension leads to a well-characterized sequence of events, and ultimately to vascular decompensation, due to a continuous increase in peripheral vascular resistance. Cardiovascular adaptation to hemorrhagic shock is dynamically controlled by endocrine and local paracrine factors, such as NO, in part to compensate for the sudden hypovolemia²⁰. NO production keeps the vasculature relaxed, regulating blood pressure and tissue perfusion. The effects of perfusion on gas exchange are one of the most important features of NO supplementation in pulmonary physiology. As changes in the distribution of blood flow to different areas of the lung, and adjustments of vessel diameter in the respective regions of the lung, could rapidly affect gas exchange. NO is a key molecule for fast response, which links alveolar ventilation to local lung perfusion. Hamster's environmental adaptations are responsible, for their low arterial PO₂, their natural environment are in burrows, either permanently or intermittently, and often demonstrate structural and functional differences from surface dwelling mammals. The so marked improvement in gas exchange, decrease in oxygen content and increase in PCO₂ seen in the NO-group can be the result of improved ventilation-perfusion, as NO has the singular property of preferentially dilating vascular segments located in ventilated areas²¹. Additionally, NO modulates synaptic signaling, cellular defense and mitochondria oxygen utilization²². Our results indicate that restoring intravascular NO concentrations by replenishing/increasing intravascular NO availability by treatment with NO-nps reduces vascular complications resulting from hemorrhage shock. The exogenous NO released induced by treatment with 5 mg/kg of NO-nps was evidenced by significant vasodilation, metHb formation, and increase in plasma nitrite and nitrate.

In our study, NOS inhibition caused an increase of vascular resistance, decreased heat rate, and increased mortality. Similarly, a clinical study using L-NAME was discontinued because the treatment showed higher mortality²³. Other NOS inhibitors were clinically evaluated to prevent hypotension; with poor results, as they increased ischemia and cardiovascular collapse^{24, 25}. Several investigations suggest that inducible NOS (iNOS) is responsible in part for producing organ injury after hemorrhage and resuscitation²⁶⁻²⁸. Until selective iNOS inhibitors are available, these findings should be analyzed with discretion. Moreover, hypoxia and ischemia during shock initiate tissue adaptive responses through transcriptional activation of several factors, such as HIF-1, which requires NO as a regulatory molecule. Traditionally, NO has been thought to be a signal, exclusively via its stimulation of guanylyl cyclase, inducing an increase in intracellular cGMP levels and, in turn, the allosteric activation of cGMP dependent kinase. Moreover, in addition to the activation of PKG, the cGMP-dependent effects can also be mediated by other proteins whose activities are allosterically modified by cGMP, and via cross-activation of cAMP-dependent kinase. In previous work, we have demonstrated that NO-np vascular effect effects are mediated by guanylyl cyclase, and can be enhanced by phosphodiesterase inhibitors and blocked by irreversible heme-site inhibitors of soluble guanylyl cyclase¹³. Thus, acute inhibition of phosphodiesterase activity, by drugs such as sildenafil, combined with NO-np represents an attractive therapeutic strategy to manipulate systemic vascular tone.

Exogenous NO in the form of NO-nps treatment following severe hemorrhage, partially recovered systemic and microvascular conditions, via reduction of precapillary resistance when compared to animals treated with Control-nps or the Vehicle. Results show a strong correlation between cardiac stability and microvascular flow. Mechanistically, it is expected that intravascular NO supplementation in the form of NO-np reduces MAP by decreasing PVR. Conversely, the experimental results indicate that NO-np increased sustained higher MAP and HR during the observation period. Central cardiac effects could reflect increases in cardiac output mediated by lower arteriolar after-load, cardiac filling and increased preload (venous return), due to NO vasodilatory properties and cardiac chronotropic effects²⁸. NO is a regulator of cardiac function through indirect vascular-dependent mechanisms and by direct action on the myocardium as a paracrine autacoid involved in autonomic control and contractility²⁹. This effect appears to be NO concentrations dependent, as physiological levels of NO increases HR via activation of hyperpolarization induced inward current, and is less pronounced at high concentrations of NO^{30, 31}. The exogenous NO released from the NO-nps maintained central hemodynamic function by preventing rhythm disturbances, implying a protective role of NO on cardiac over-drive and pacing³². NO has effects in cardiomyocytes, which are cyclic GMP-dependent and other effects that are cyclic GMP-independent³⁴. Heart muscle cells express the soluble isoform of guanylyl cyclase that catalytically increases intracellular levels of cyclic GMP in response to stimulation by pure NO, or GSNO³⁴. Moreover, heart rate can directly affect cardiac output, if stroke volume remains constant, therefore results observed with NO-np can be the result of sustaining higher heart rates and potentially, cardiac output, which ultimately increase systemic oxygen delivery. These results show that the principal factor in ensuring hemodynamic restoration by NO-nps is not related to a volume effect since hematocrits did not differ between groups.

Clinically, vasodilators as adjunct therapy to fluid resuscitation were shown to be effective in restoring the microvascular function³⁵. Nitroglycerin with adequate volume support, has been shown to reinstate capillary flow and restore sublingual microcirculation³⁶. This work explores the connection between cardiovascular collapse post hemorrhagic shock, and reduced NO bioavailability, by increasing the NO bound to low molecular weight thiols (1 mg/kg nitrosoglutathione, GSNO) prior to the hemorrhage³⁶. Since GSNO only is stable in vivo at basal conditions for a limited period of time, its application is limited. Therefore, our current study goes beyond previous results with GSNO administrated prior hemorrhage, to a therapy applicable 10 min post the hemorrhage based on NO-np. The most important characteristic to supplement NO post hemorrhage is the intravascular controlled release of moderate levels of NO to mimic physiologically generated NO, a characteristic currently achievable with NO-np. NO donors, such as nitroglycerin, an organic nitrate, and clinically approved, is short acting, involves specific enzymes and reducing agents, and it is not suitable for a controlled and sustained release. Other NO donors, where the NO is bound to a nucleophile, and the NO is liberated after spontaneous decomposition, were not suitable as therapies post hemorrhage. NO-nanoparticles release pure NO, freely soluble in physiological solution, and do not require cofactors to facilitate NO release. The current results indicate that treatment with 5 mg/kg NO-nps, prevented hemorrhagic shock, induced vascular decompensation, and set the proof of the concept that NO supplementation after hemorrhage will reduce the risks of cardiovascular collapse. To date, there are no is definitive treatments for addressing massive blood loss on the battlefield, and existing therapies for severe hemorrhage are particularly limited. NO-nps treatment maintains perfusion reducing hemorrhagic shock sequelae, with an infusion volume of less than 2.5% of the shed blood volume. NO-np is the logical progression of the concepts initially defined with GSNO, as GSNO is compromised by thermal decomposition, photolysis and in vivo catalytic decomposition. The reduction in weight therapy provided by NO-np relative to fluid resuscitation can greatly reduce logistical burdens, and the stability of NO-nps for months exposed to air at room temperature, facilitates its application in multiple scenarios.

In conclusion, this study shows that severe hemorrhage induces vascular decompensation, in part, due to low availability of NO during post hemorrhage, and that exogenous NO during this stage can prevent circulatory arrest. Exogenous NO in the from of NO-nps attenuated microvascular complications during hypovolemia, which targets a pivotal protective function by maintaining tissue perfusion, assuring wash-out of metabolic residues and thereby preventing future damage during reperfusion. Mechanistically, partial preservation of microvascular perfusion during hypovolemic shock due to increased NO bioavailability, acts by regulating vascular tone and pressure redistribution, and maintaining capillary pressure and metabolite exchange. Lastly, NO has a fundamental signaling role in cellular function with implications in cardiac chronotropic function, which appears to be jeopardized as hypovolemic shock is established.

### REFERENCES FOR EXAMPLE 3

1. Bellamy R F. The causes of death in conventional land warfare: implications for combat casualty care research. Mil Med 149: 55-62, 1984.
2. Pope AM and Institute of Medicine (U.S.). Fluid resuscitation: state of the science for treating combat casualties and civilian injuries. Washington, D.C.: National Academy Press, 1999.
3. Bacter C R, Canizaro P C, Carrico C J, and Shires G T. Fluid resuscitation of hemorrhagic shock. Postgrad Med 48: 95-99, 1970.
4. Burris D, Rhee P, Kaufmann C, Pikoulis E, Austin B, Eror A, et al. Controlled resuscitation for uncontrolled hemorrhagic shock. J Trauma 46: 216-223, 1999.
5. Dubick M A and Atkins J L. Small-volume fluid resuscitation for the far-forward combat environment: current concepts. J Trauma 54: S43-45, 2003.
6, Kristiansson M, Soop M, Saraste L, and Sundqvist KG. Cytokines in stored red blood cell concentrates: promoters of systemic inflammation and simulators of acute transfusion reactions? Acta Anaesthesiol Scand 40: 496-501, 1996.
7, Ignarro L J, Byrns R E, Buga G M, and Wood K S. Endothelium-derived relaxing factor from pulmonary artery and vein possesses pharmacologic and chemical properties identical to those of nitric oxide radical, Circ Res 61: 866-879, 1987.
8. Salazar Vazquez B Y, Martini J, Chavez Negrete A, Cabrales P, Tsai A G, and Intaglietta M. Microvascular benefits of increasing plasma viscosity and maintaining blood viscosity: counterintuitive experimental findings. Biorheology 46: 167-179, 2009.
9. Revers L M, Braam B, Post J A, van Zonneveld A J, Rabelink T J, Koomans H A, et so al. Tetrahydrobiopterin, but not L-arginine, decreases NO synthase uncoupling in cells expressing high levels of endothelial NO synthase. Hypertension 47: 87-94, 2006.
10. Allen B W, Demchenko I T, and Piantadosi C A. Two faces of nitric oxide: implications for cellular mechanisms of oxygen toxicity, J Appl Physiol 106: 662-667, 2009.
11. Lancaster JR. Simulation of the diffusion and reaction of endogenously produced nitric oxide. Proc Natl Acad Sci USA 91: 8137-8141, 1994.
12. Friedman A J, Han G, Navati M S, Chacko M, Gunther L, Alfieri A, et al. Sustained release nitric oxide releasing nanoparticles: characterization of a novel delivery platform based on nitrite containing hydrogel/glass composites. Nitric Oxide 19: 12-20, 2008.
13. Cabrales P, Han G, Roche C, Nacharaju P, Friedman A J, and Friedman J M. Sustained release nitric oxide from long-lived circulating nanoparticles. Free Radic Biol Med, 2010.
14. Colantuoni A, Bertuglia S, and Intaglietta M. Quantitation of rhythmic diameter changes in arterial microcirculation. Am J Physiol 246: H508-H517, 1984.
15. Altman DG and Bland J M. Statistics notes: How to randomise. BMJ 319: 703-704, 1999.
16. Winterbourn CC. Reaction of superoxide with hemoglobin. CRC Handbook of Methods for Oxygen Radical Research. Boca Raton, Fla.: CRC Press, 1985.
17. Intaglietta M, Silverman N R, and Tompkins W R. Capillary flow velocity measurements in vivo and in situ by television methods. Microvasc Res 10: 165-179, 1975,
18. Lipowsky H H and Zweifach BW. Application of the “two-slit” photometric technique to the measurement of microvascular volumetric flow rates. Microvasc Res 15: 93-101, 1978.
19. Intaglietta M and Tompkins W R. Microvascular measurements by video image shearing and splitting. Microvasc Res 5: 309-312, 1973.
20. Schadt J C and Ludbrook J. Hemodynamic and neurohumoral responses to acute hypovolemia in conscious mammals. Am J Physiol 260: H305-318, 1991.
21. Rossaint R, Falke K J, Lopez F, Slama K, Pison U, and Zapol WM. Inhaled nitric oxide for the adult respiratory distress syndrome. N Engl J Med 328: 399-405, 1993.
22. Moncada S and Erusalimsky JD. Does nitric oxide modulate mitochondrial energy generation and apoptosis? Nat Rev Mol Cell Biol 3: 214-220, 2002.
23. Lopez A, Lorente J A, Steingrub J, Bakker J, McLuckie A, Willatts 5, et al. Multiple-center, randomized, placebo-controlled, double-blind study of the nitric oxide synthase inhibitor 546C88: effect on survival in patients with septic shock. Crit Care Med 32: 21-30, 2004.
24. Md S, Moochhala S M, and Siew-Yang K L. The role of inducible nitric oxide synthase inhibitor on the arteriolar hyporesponsiveness in hemorrhagic-shocked rats. Life Sci 73: 1825-1834, 2003.
25, Thiemermann C, Szabo C, Mitchell JA, and Vane JR. Vascular hyporeactivity to vasoconstrictor agents and hemodynamic decompensation in hemorrhagic shock is mediated by nitric oxide. Proc Natl Acad Sci USA 90: 267-271, 1993.
26. Banta S, Yokoyama T, Berthiaume F, and Yarmush ML. Effects of dehydroepiandrosterone administration on rat hepatic metabolism following thermal injury. J Surg Res 127: 93-105, 2005.
27. Shimizu T, Tani T, Endo Y, Hanasawa K, Tsuchiya M, and Kodama M. Elevation of plasma peptidoglycan and peripheral blood neutrophil activation during hemorrhagic shock: plasma peptidoglycan reflects bacterial translocation and may affect neutrophil activation. Crit Care Med 30: 77-82, 2002.
28. Menezes J M, Hierholzer C, Watkins S C, Billiar T R, Peitzman A B, and Harbrecht B G. The modulation of hepatic injury and heat shock expression by inhibition of inducible nitric oxide synthase after hemorrhagic shock. Shock 17: 13-18, 2002.
29. Fatehi-Hassanabad Z and Fatehi M. Characterisation of some pharmacological effects of the venom from Vipera lebetina. Toxicon 43: 385-391, 2004.
30. Han X, Kobzik L, Zhao Y Y, Opel D J, Liu W D, Kelly R A, et al. Nitric oxide regulation of atrioventricular node excitability. Can J Cardiol 13: 1191-1201, 1997.
31. Herring N, Rigg L, Terrar DA, and Paterson DJ. NO-cGMP pathway increases the hyperpolarisation-activated current, I(f), and heart rate during adrenergic stimulation. Cardiovasc Res 52: 446-453, 2001.
32. Massion PB and Balligand J L. Modulation of cardiac contraction, relaxation and rate by the endothelial nitric oxide synthase (eNOS): lessons from genetically modified mice. J Physiol 546: 63-75, 2003.
33. Khadour FH, O'Brien D W, Fu Y, Armstrong P W, and Schulz R. Endothelial nitric oxide synthase increases in left atria of dogs with pacing-induced heart failure. Am J Physiol 275: H1971-1978, 1998.
34. Mayer B, Pfeiffer 5, Schrammel A, Koesling D, Schmidt K, and Brunner F. A new pathway of nitric oxide/cyclic GMP signaling involving S-nitrosoglutathione. J Biol Chem 273: 3264-3270, 1998.
35. De Backer D, Creteur J, Dubois M J, Sakr Y, and Vincent J L. Microvascular alterations in patients with acute severe heart failure and cardiogenic shock. Am Heart J 147: 91-99, 2004.
36. Spronk P E, Ince C, Gardien M J, Mathura K R, Oudemans-van Straaten H M, and Zandstra D F. Nitroglycerin in septic shock after intravascular volume resuscitation. Lancet 360: 1395-1396, 2002.
37. Cabrales P, Tsai AG, and Intaglietta M. Exogenous nitric oxide induces protection during hemorrhagic shock. Resuscitation 80: 707-712, 2009.

1. A method for delivering a nitric oxide (NO) molecule or precursor thereof to a vascular compartment of a subject comprising: administering to the subject, a NO particle formulation, via an intravascular, intraperitoneal, or intramuscular administration, in an amount sufficient to induce vascular smooth muscle relaxation thereby delivering the NO molecule or precursor thereof to the vascular compartment of the subject, wherein the NO particle formulation comprises NO attached to a nanoparticle or microparticle.

2. A method for enlarging the diameter of blood vessels by delivering a NO molecule or precursor thereof in the vascular compartment of blood vessels by the method of claim 1.

3. A method for increasing perfusion and oxygenation of blood vessels by delivering a NO molecule or precursor thereof to the vascular compartment of blood vessels by the method of claim 1.

4. A method for decreasing blood pressure in a subject by delivering a NO molecule or precursor thereof to the vascular compartment of blood vessels of the subject by the method of claim 1.

5. A method for decreasing macrophage activation in a subject by delivering a NO molecule or precursor thereof to the vascular compartment of blood vessels of the subject by the method of claim 1.

6. A method of alleviating the symptoms of peripheral vascular disease in a subject suffering therefrom, by delivering a NO molecule or precursor thereof to the vascular compartment of blood vessels of the subject by the method of claim 1.

7. The method of claim 1, wherein the particle further comprises a detection or therapeutic agent bound to the surface of the particle via the terminal hydroxyl groups.

8. The method of claim 7, wherein the therapeutic agent is a cytotoxic agent.

9. (canceled)

10. The method of claim 7, wherein the therapeutic agent is selected from the group consisting of streptokinase, tissue plasminogen activator, plasmin, urokinase, a tissue factor protease inhibitor, anticoagulant protein, a metalloproteinase inhibitor, an anti-inflammatory agent, steroids, analgesics, local anesthetics, antibiotic agents, chemotherapeutic agents, immunosuppressive agents, antiproliferative agents, antimitotic agents, angiogenic agents, antipsychotic agents, central nervous system (CNS) agents; fibrinolytic agents, growth factors, and antibodies.

11. The method of claim 7, wherein said detection agent is a fluorescent polypeptide, paramagnetic isotope, a heavy metal, or a radioisotope.

12. (canceled)

13. The method of claim 1, wherein the NO particle formulation comprises a NO molecule or precursor thereof encapsulated in a matrix having a chitosan or equivalent thereof, a polyethylene glycol (PEG), a tetra-methoxy-ortho-silicate (TMOS) molecule.

14. (canceled)

15. (canceled)

16. The method of claim 1, wherein the NO precursor is selected from the group consisting of organic nitrites and nitrates.

17. The method of claim 1, wherein the formulation comprises a nanoparticle having a size range of about 5 to 1000 nanometers (nm) in diameter.

18. The method of claim 17, wherein the formulation comprises a nanoparticle having a size range of from about 100 to 800 nanometers in diameter.

19. The method of claim 17, wherein the formulation comprises a nanoparticle having a size range of between 50 nm and 300 nm.

20. The method of claim 17, wherein the formulation comprises a nanoparticle having a size range of between 20 nm and 200 nm.

21-24. (canceled)

25. The method of claim 1, wherein the subject is selected from a group consisting of humans, non-human primates, rodents, guinea pigs, rabbits, sheep, pigs, goats, cows, horses, dogs, cats, birds, and fish.

26. A method of inhibiting cardiovascular disease in a subject by delivering NO in the vascular compartment of blood vessels of the subject by the method of claim 1.

27. The method of claim 26, wherein the cardiovascular disease causes plaque rupture, plaque erosion, acute coronary syndrome, stroke, transient ischemia attack, heart attack, angina, unstable angina, thrombosis, myocardial infarction, ischemic heart disease, peripheral artery disease, or transplantation-induced sclerosis.

28. The method of claim 1, wherein the amount of the NO particle formulation so administered is from about 0.1 to 1000 mg/kg of body weight.
